(19) United States
(12) Patent Application Publication
(10) Pub. No.: US 2019/0153430 A1
(43) Pub. Date: May 23, 2019

(54) METHOD FOR GENOME EDITING

(71) Applicant: KYOTO UNIVERSITY, Kyoto (JP)

(72) Inventors: Knut WOLTJEN, Kyoto (JP); Shin-Il KIM, Kyoto (JP); Tomoko MATSUMOTO, Kyoto (JP)

(73) Assignee: KYOTO UNIVERSITY, Kyoto (JP)

(21) Appl. No.: 16/322,924

(22) PCT Filed: Aug. 2, 2017
(86) PCT No.: PCT/IB2017/054736

§ 371 Date: Feb. 1, 2019

Related U.S. Application Data
(60) Provisional application No. 62/370,047, filed on Aug. 2, 2016.

(51) Int. Cl. C12N 15/10; C12N 9/22; C12N 15/11

(52) U.S. Cl. CPC C12N 15/102; C12N 15/11; C12N 9/22

(57) ABSTRACT

The present invention provides a method of producing a cell having a scarless genome sequence wherein an exogenous nucleic acid sequence inserted into a targeted region in the genome is completely excised, wherein the exogenous nucleic acid sequence comprises a nucleic acid sequence homologous to a genome sequence in the targeted region at each end and one or more sequence-specific nuclease-recognizing site(s) between the two homologous nucleic acid sequences, and wherein the method comprises: (1) introducing the sequence-specific nuclease or a nucleic acid encoding the same into a host cell having a genome sequence into which the exogenous nucleic acid sequence is inserted; and (2) culturing the cell obtained in step (1), thereby causing double-strand break at the sequence-specific nuclease-recognizing site(s) and the subsequent microhomology-mediated end joining or single-strand annealing between the resulting broken ends that contain the homologous nucleic acid sequences to generate a cell having a scarlessly reverted genome sequence in which the exogenous nucleic acid sequence is completely excised from the targeted region.

### TECHNICAL FIELD

The present invention relates to a novel method for gene editing. More particularly, the present invention relates to a method for scarless excision of a transgene such as selectable marker gene from a host genome using microhomology-mediated end joining or single-strand annealing. The present invention also relates to production of a cell having a mutation in a targeted region in its genome and an isogenic cell without the mutation, using the above-mentioned method, and the like.

### BACKGROUND ART

Functional genomics relies on gene targeting to create or revert mutations implicated in regulating protein activity or gene expression. This methodology has advanced greatly across species through the development of designer nucleases such as ZFNs, TALENs, and CRISPR/Cas9 (Kim and Kim, Nature reviews Genetics 15, 321-334, 2014; Sakuma and Woltjen, Dev Growth Differ 56, 2-13, 2014), with CRISPR/Cas9 taking the lead due to the simplicity of programmable sgRNA cloning, coupled with efficient and reproducible genomic cleavage. Despite differences in experimental design and DNA cleavage mechanism, all engineered nucleases function by generating targeted double strand breaks (DSBs) to induce cellular repair pathways. Error-prone repair via non-homologous end joining (NHEJ) is typically sufficient for gene disruption, while homology directed repair (HDR) can be usurped with custom template DNA that acts as a donor in the repair of targeted double-strand breaks, allowing for more specific gene editing. These advances are of particular interest in the field of human genetics for disease modelling, where gene targeting in human induced pluripotent stem cells (iPSCs) with nucleases enables the original patient iPSC line to act as an isogenic control (Hockemeyer and Jaenisch, Cell stem cell 18, 573-586, 2016).

Although recent advances in nuclease technology have respectably improved gene targeting efficiencies for human embryonic stem cells (ESCs) or iPSCs, the deposition of single nucleotide variations which mimic or correct patient mutations remains difficult without a robust means for enrichment and selection, such that positive selection for antibiotic resistance markers remains a staple in gene targeting (Capecchi, Nature reviews Genetics 6, 507-512, 2005). Moreover, positive selection provides a method for generating clonal populations with minimal effort.

For genome editing by conventional gene targeting with positive selection, scarless excision of the antibiotic selection marker is a critical step, yet remains non-trivial using current methods. Methods such as Cre-loxP recombination (Davis et al., Nature protocols 3, 1550-1558, 2008), and more recently excision-prone transposition (Firth et al., Cell reports 12, 1385-1390, 2015) have been shown to remove selection cassettes after their utility is expended. However, these methods are fraught with complications such as residual recombinase sites (Meier et al., FASEB journal: official publication of the Federation of American Societies for Experimental Biology 24, 1714-1724, 2010), low excision frequencies, and potential for cassette re-integration (Ye et al., Proceedings of the National Academy of Sciences of the United States of America 111, 9591-9596, 2014). Alternative methods to achieve scarless excision must therefore be sought.

Within the repertoire of endogenous cellular repair pathways, microhomology-mediated end joining (MMEJ) and single-strand annealing (SSA), are underappreciated mechanisms for repairing DSBs. MMEJ and SSA are Ku-independent pathways that employ naturally-occurring microhomology (μH) of 5-25 bp or longer (>30 bp) homology, respectively, occurring on either side of the DSB to mediate end joining (McVey and Lee, Trends in genetics: TIG 24, 529-538, 2008). The outcome of MMEJ is a reproducible deletion of intervening sequences while retaining one copy of the μH. For this reason, MMEJ is normally considered to be mutagenic, because of an overall loss of genetic information by precision deletion.

### SUMMARY OF INVENTION

In the present invention, the inventors addressed the issue of high-fidelity excision by recruiting MMEJ. Using standard donor vector design where a point mutation is juxtaposed with a positive selection cassette, the inventors went on to engineer μH to flank the selection cassette through a simple PCR-generated overlap in the left and right homology arms. After positive selection for gene targeting, the inventors introduced DSBs using validated and standardized CRISPR/Cas9 protospacers nested between the cassette and μH, stimulating the cell to employ MMEJ and scarlessly excise the cassette, leaving behind only the designer point mutation at the locus. Moreover, employing imperfect microhomology, the inventors demonstrated that it is possible to produce isogenic mutant and control iPSC lines from the same experiment, addressing a current concern in the field over the effects of nuclease and cell culture manipulations. Finally, the inventors employed the technique to develop an iPSC model for the HPRT_{Munich} partial enzyme deficiency, discovered in a patient presenting with gout caused by hyperuricemia (Wilson et al. J Biol Chem 256, 10306-10312, 1981), and use measures of cellular metabolism to establish a consistent molecular phenotype between iPSC clones. We expect this technique to have broad applications, even beyond scarless iPSC genome editing. While we used MMEJ as working examples, SSA shares genetic requirements in common with MMEJ and is also applicable.

That is, the present invention provides:

[1] a method of producing a cell having a scarless genome sequence wherein an exogenous nucleic acid sequence inserted into a targeted region in the genome is completely excised,

wherein the exogenous nucleic acid sequence comprises a nucleic acid sequence homologous to a genome sequence in the targeted region at each end and one or more sequence-specific nuclease-recognizing site(s) between the two homologous nucleic acid sequences, and wherein the method comprises:

(1) introducing the sequence-specific nuclease or a nucleic acid encoding the same into a host cell having a genome sequence into which the exogenous nucleic acid sequence is inserted; and

(2) culturing the cell obtained in step (1),

thereby causing double-strand break at the sequence-specific nuclease-recognizing site(s) and the subsequent microhomology-mediated end joining or single-strand annealing between the resulting broken ends that contain the homologous nucleic acid sequences to generate a cell having a scarlessly reverted genome sequence in which the exogenous nucleic acid sequence is completely excised from the targeted region;

[2] the method according to [1] above, wherein the exogenous nucleic acid sequence comprises two or more sequence-specific nuclease-recognizing sites and two of them are located substantially adjacent to the two homologous nucleic acid sequences, respectively, and an exogenous gene is inserted between the two sequence-specific nuclease-recognizing sites;

[3] the method according to [2] above, wherein the exogenous gene is a selectable marker gene;

[4] the method according to any one of [1]-[3] above, wherein either or both of the homologous nucleic acid sequences have a mutation in the corresponding endogenous genome sequence;

[5] the method according to [4] above, wherein both of the homologous nucleic acid sequences have the same mutation, thereby generating a cell having a genome sequence with the mutation in the targeted region;

[6] the method according to [4] above, wherein either of the homologous nucleic acid sequences has a mutation, thereby simultaneously generating a cell having a genome sequence with the mutation in the targeted region and an isogenic cell without the mutation;

[7] the method according to any one of [1]-[6] above, wherein the sequence-specific nuclease is a Zinc-finger nuclease (ZFN), a transcription activator-like effector nuclease (TALEN) or a clustered regulatory interspaced short palindromic repeats/CRISPR-associated protein (CRISPR/Cas);

[8] the method according to any one of [1]-[7] above, wherein the host cell is obtained by

introducing into a cell a nucleic acid comprising the exogenous nucleic acid sequence and, at both ends thereof, genome sequences flanking both ends of a genome sequence homologous to the homologous nucleic acid sequences, respectively,

thereby inserting the exogenous nucleic acid sequence into the targeted region of the host genome by homologous recombination;

[9] the method according to [8] above, wherein either or both of the flanking genome sequences have a mutation in the corresponding endogenous genome sequence, thereby generating a cell having a genome sequence with the mutation in the flanking genome sequence(s);

[10] the method according to [8] or [9] above, wherein the homologous recombination is mediated by sequence-specific double-strand break at a sequence-specific nuclease-recognizing site in each of the flanking genome sequences;

[11] the method according to [10] above, wherein the sequence-specific nuclease is ZFN, TALEN or CRISPR/Cas;

[12] the method according to any one of [1]-[11] above, wherein the host cell is an embryonic stem cell or an induced pluripotent stem cell;

[13] the method according to any one of [1]-[12] above, wherein the targeted region comprises a site whose mutation causes a disease;

[14] a nucleic acid for use in the method according to any one of [8]-[11] above, comprising:

(a) two nucleic acid sequences homologous to a targeted region in a host genome, wherein the 3′ end of one of the nucleic acid sequences and the 5′ end of the other nucleic acid sequence overlap; and

(b) one or more sequence-specific nuclease-recognizing site(s) between the two nucleic acid sequences of (a);

[15] the nucleic acid according to [14] above, wherein the exogenous nucleic acid sequence comprises two or more sequence-specific nuclease-recognizing sites and two of them are located substantially adjacent to the two nucleic acid sequences of (a), respectively, and an exogenous gene is inserted between the two sequence-specific nuclease-recognizing sites;

[16] a kit for use in the method according to any one of [8]-[11] above, comprising:

(a) the nucleic acid of [14] or [15] above; and

(b) one or more kinds of sequence-specific nuclease(s) specifically recognizing the sequence-specific nuclease-recognizing site(s) contained in the nucleic acid of (a), or nucleic acid(s) that encode the same;

[17] the kit according to [16] above, wherein the sequence-specific nuclease is ZFN, TALEN or CRISPR/Cas;

and the like.

The flexibility of the inventive cassette excision method could have broader applications in the elimination of foreign genetic elements for gene or cell therapy applications, and possibly even conditional gene manipulation.

### BRIEF DESCRIPTION OF DRAWINGS

FIG. 1 shows that TALEN Disruption of the HPRT1 locus is biased by MMEJ.

A. Schematic of the human HPRT1 locus with detail for segments of exon 3 and 4 (orange) including splice junctions, the HPRT1_B NC- or Avr-TALEN target sites (green), and predicted micro5W3 microhomology (blue) with the mismatched base (A/T) shown in red. Chromosome positions refer to H. sapiens GRCh38. HPRT codons are numbered above. Sequence trace of the 1383D6 iPSC genome is shown below. SD, splice donor; SA, splice acceptor.

B. Summary of repair outcomes in 6-TG^{R} clones following treatment of 1383D6 iPSCs with HPRT1_B Avr-TALENs. Individual clone sequences are listed in FIG. 5.

C. Sequence of the two most commonly observed 17 bp deletions, delta17A and delta17T.

D. Schematic of the molecular repair events leading to either delta17A or delta17T formation by MMEJ. Note that the intervening 17 bp sequence is similarly excised, despite the final outcome (A or T). microH, microhomology (blue).

FIG. 2 shows spectrum of NC-TALEN-induced mutations in human female iPSC clones.

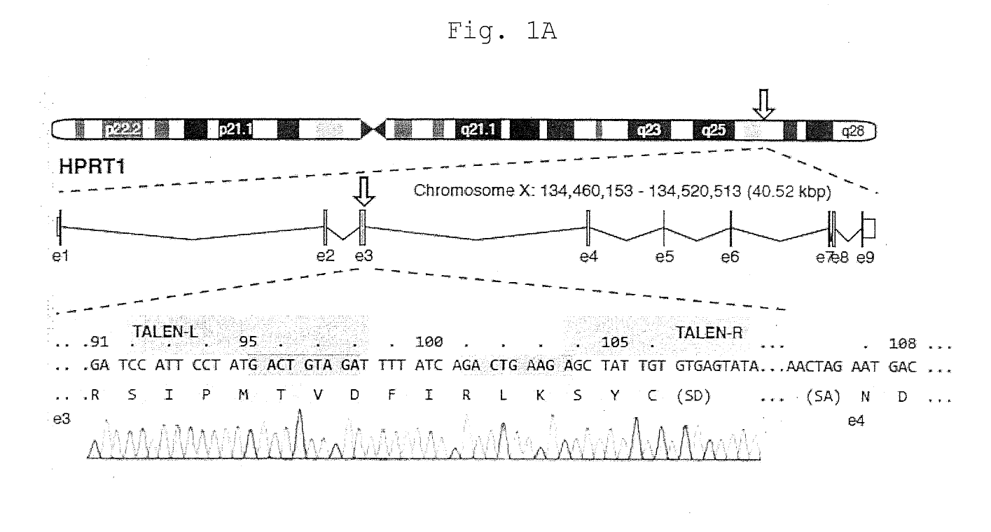

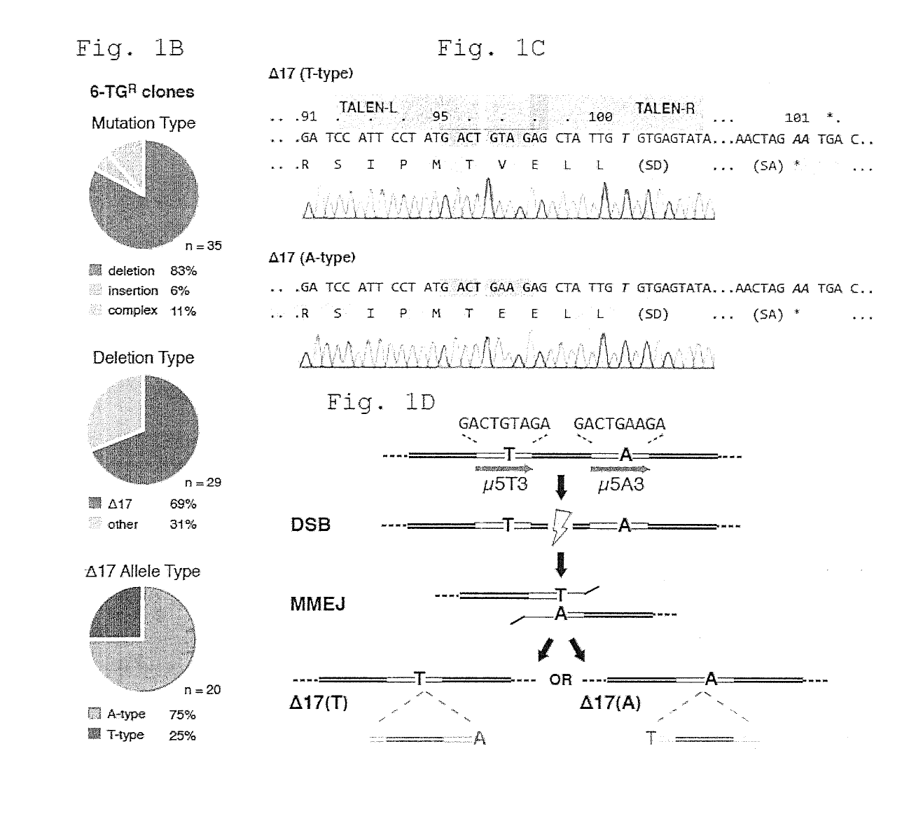

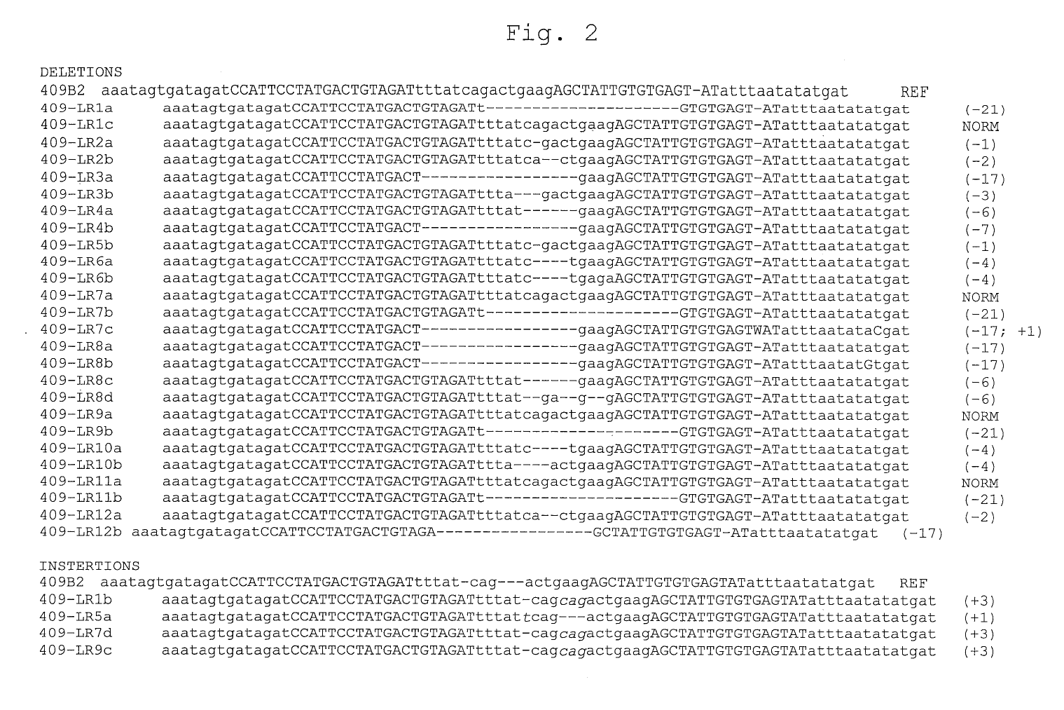

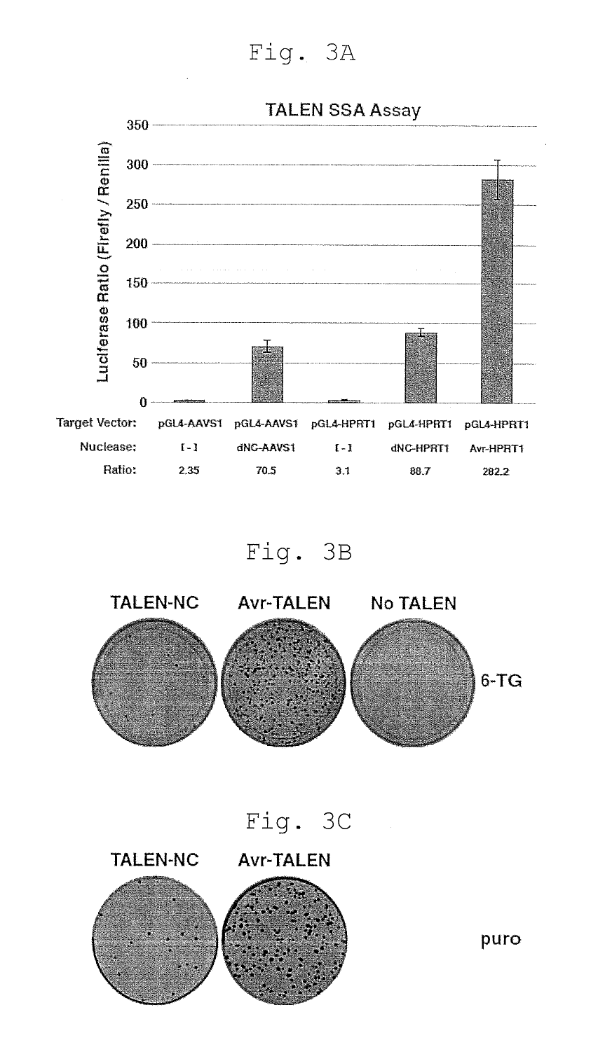

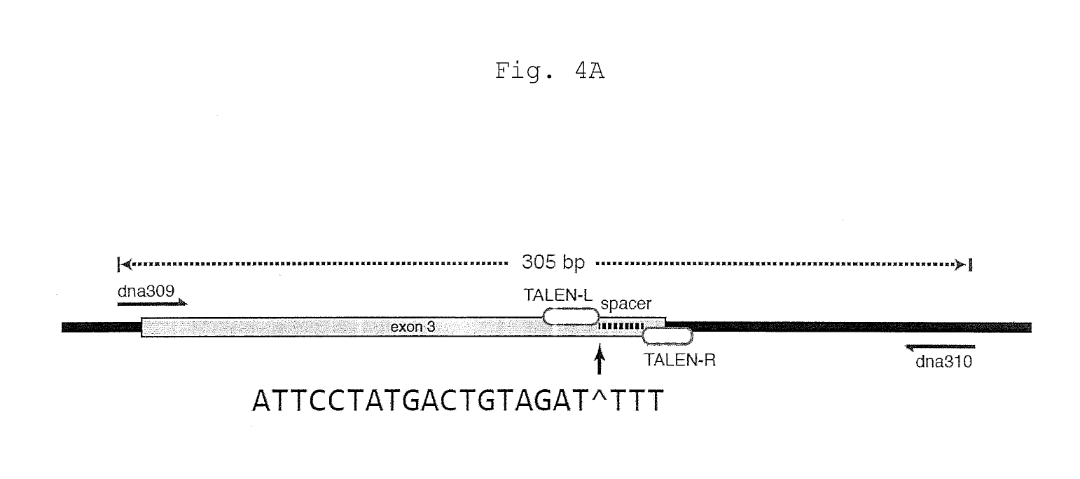

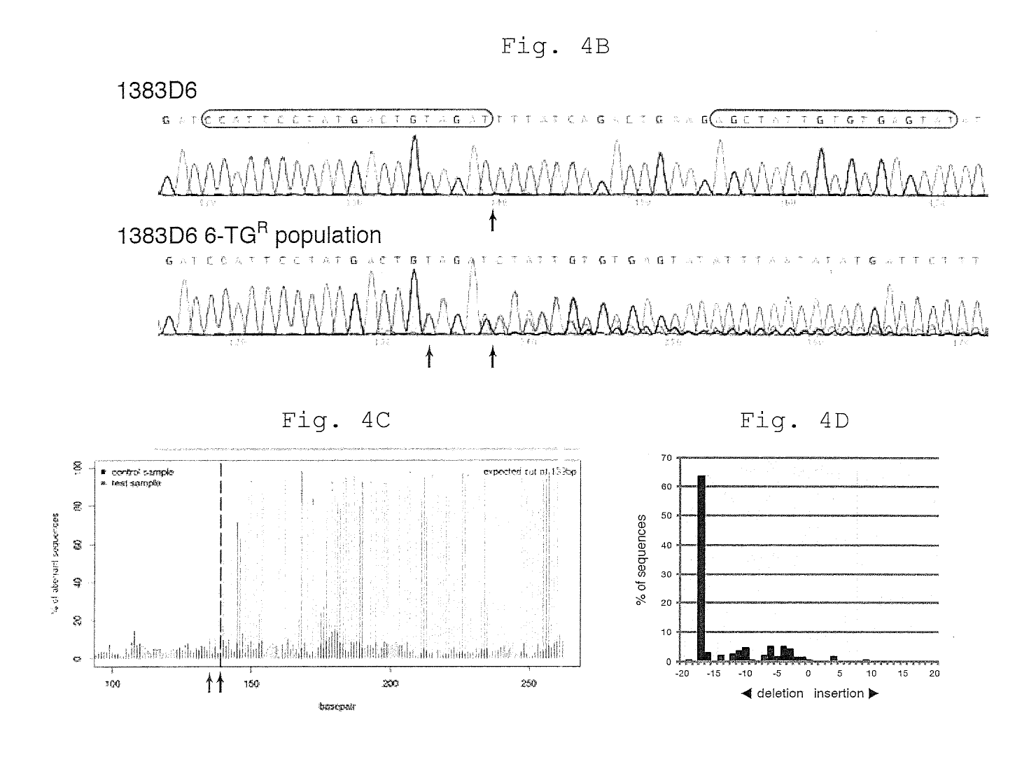

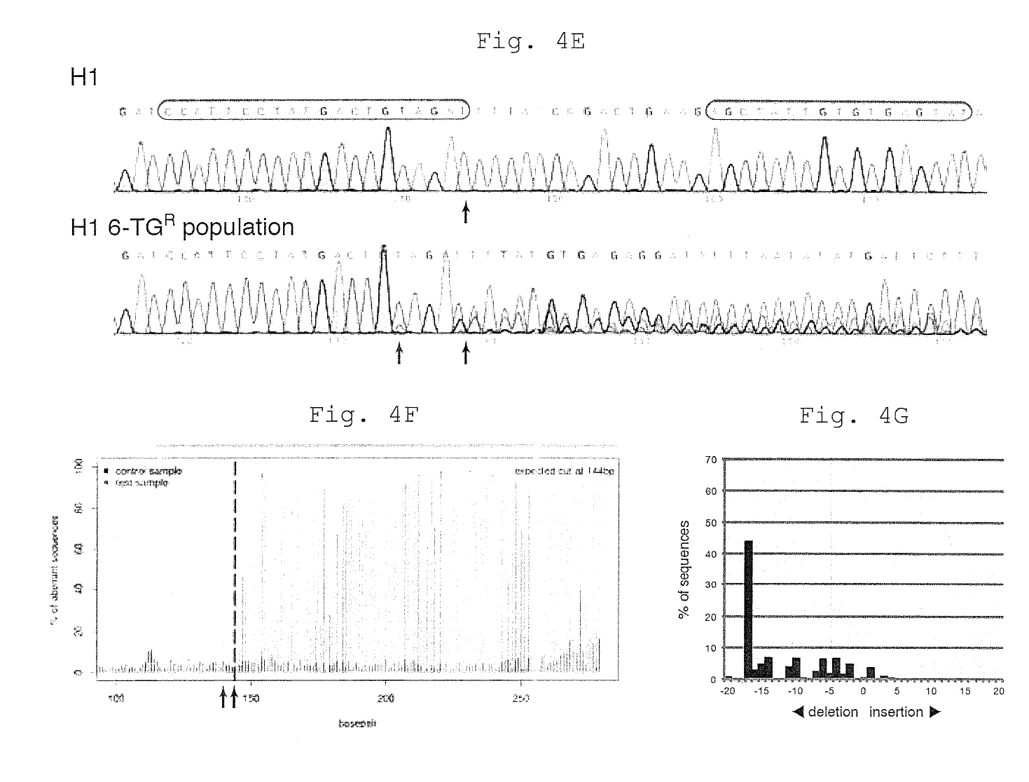

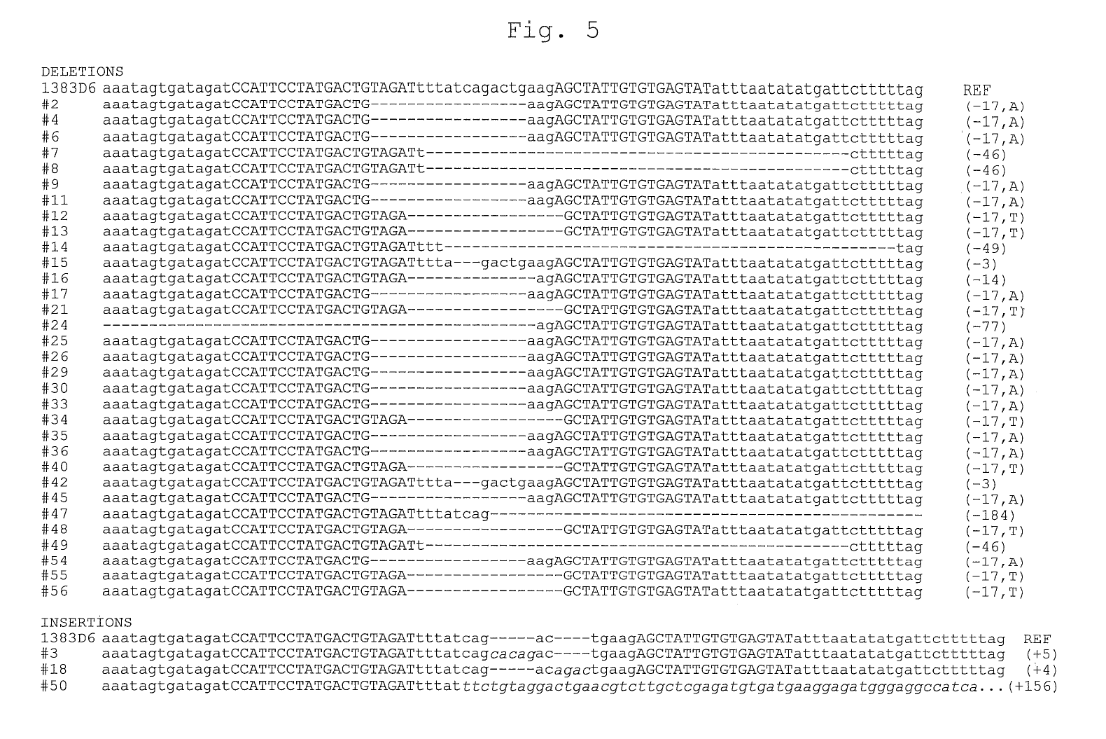

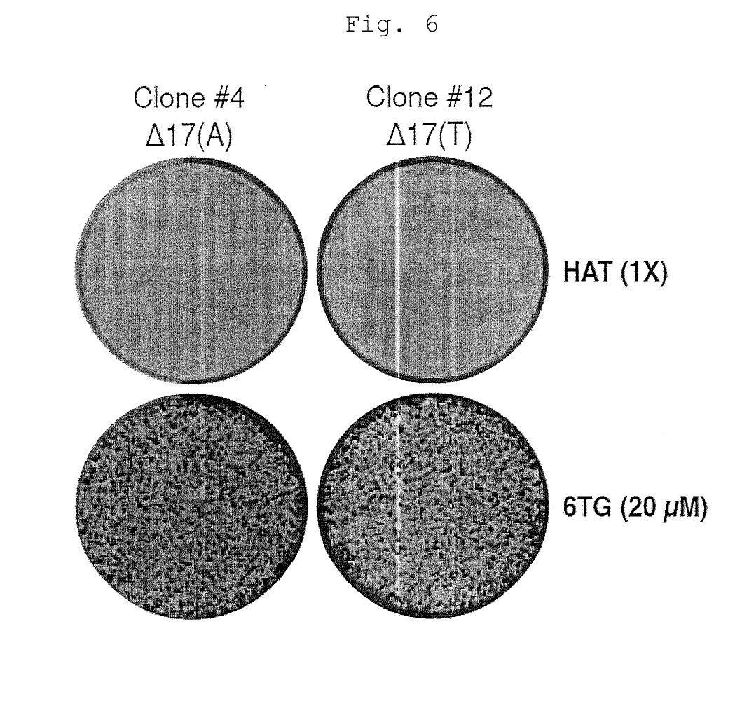

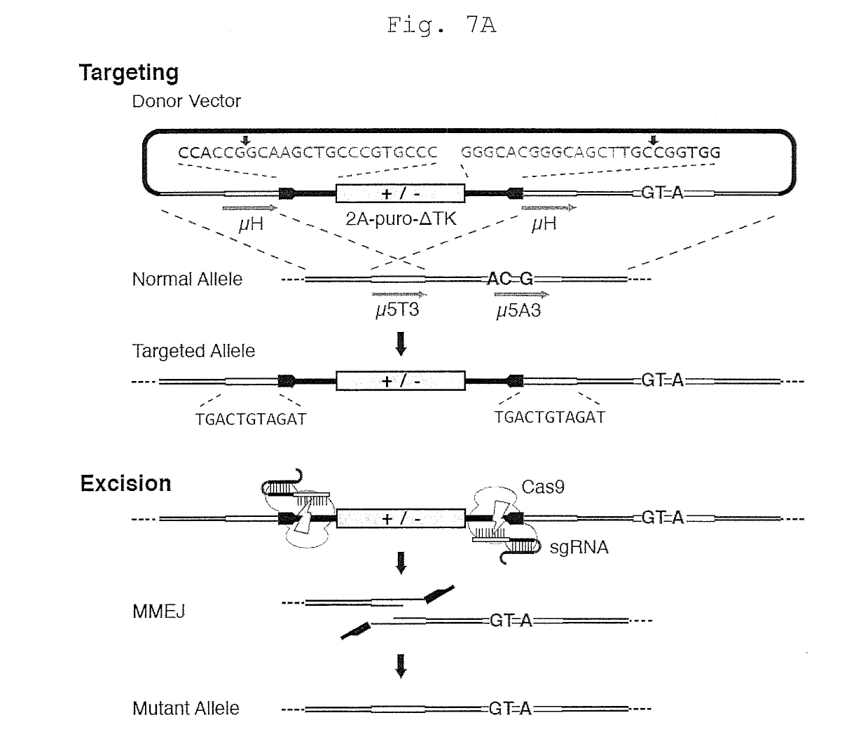

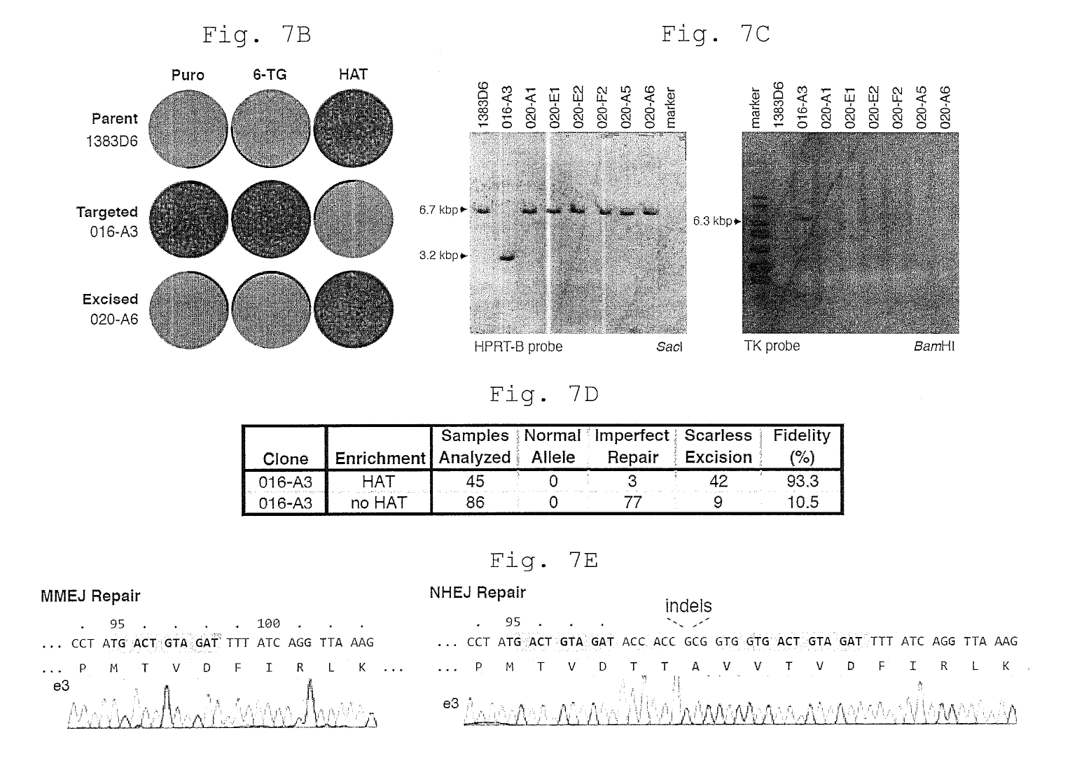

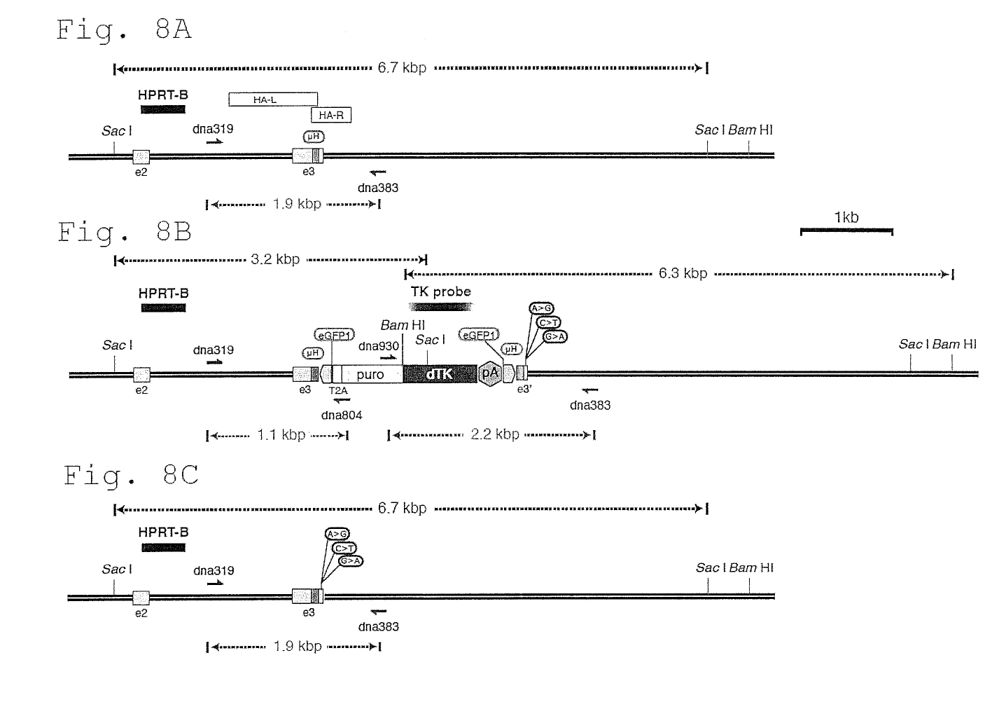

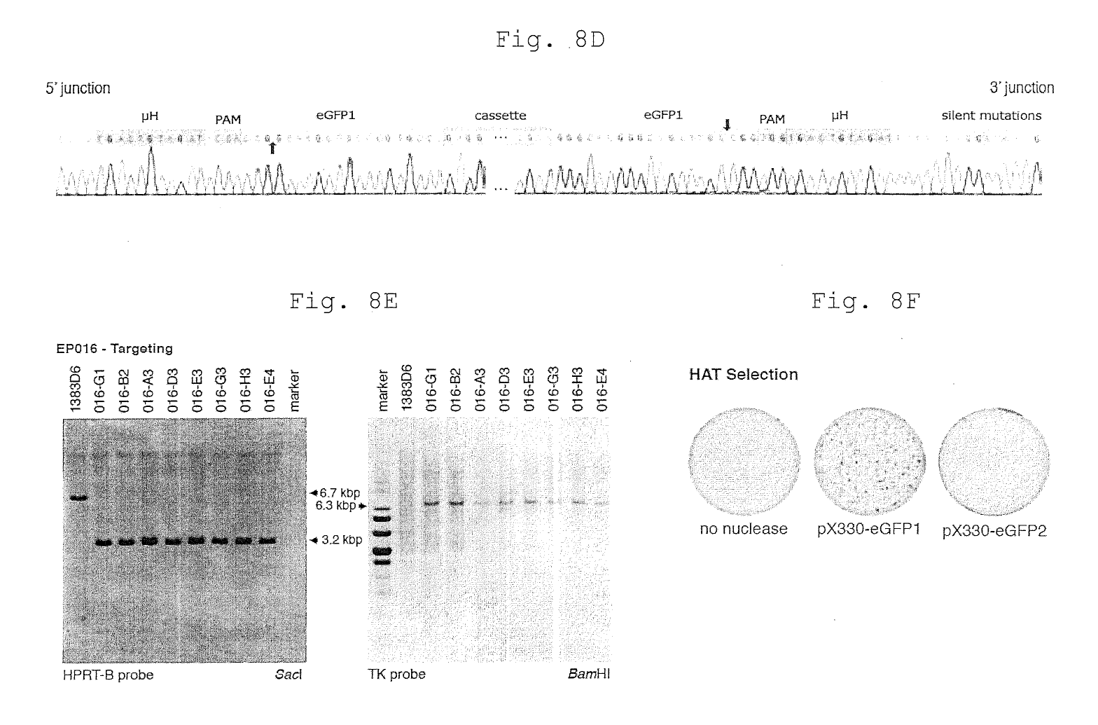

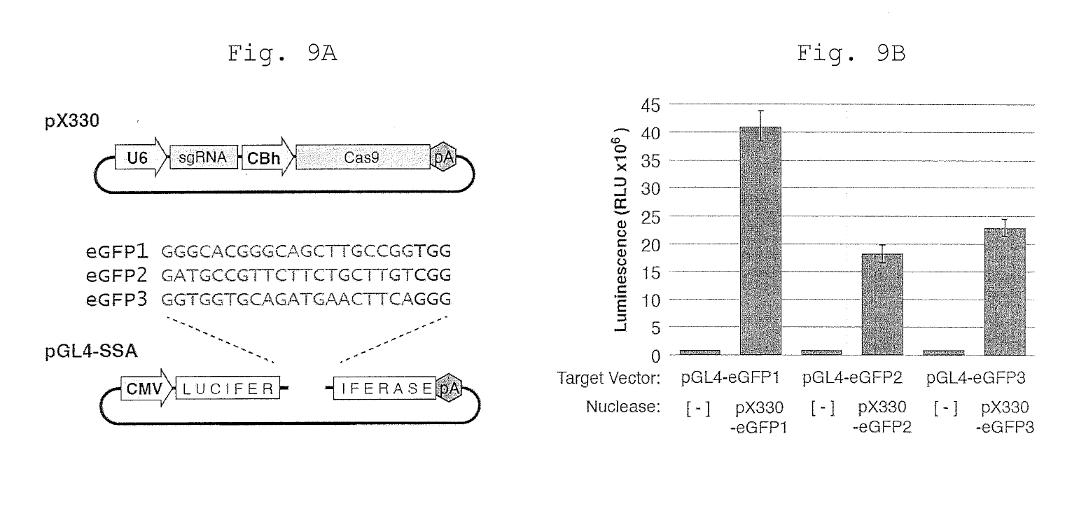

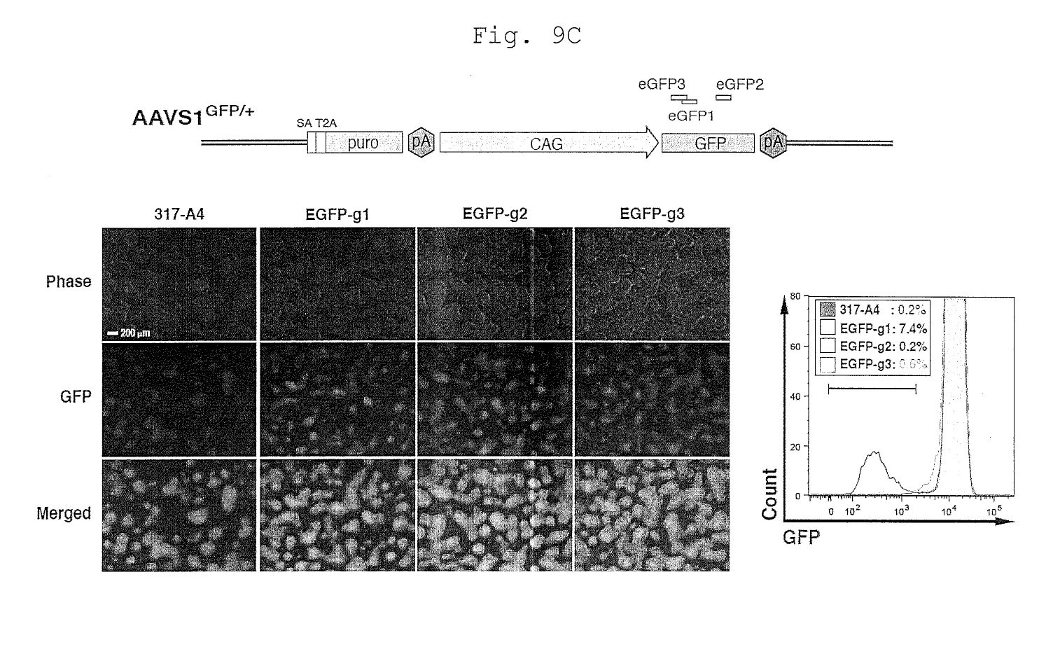

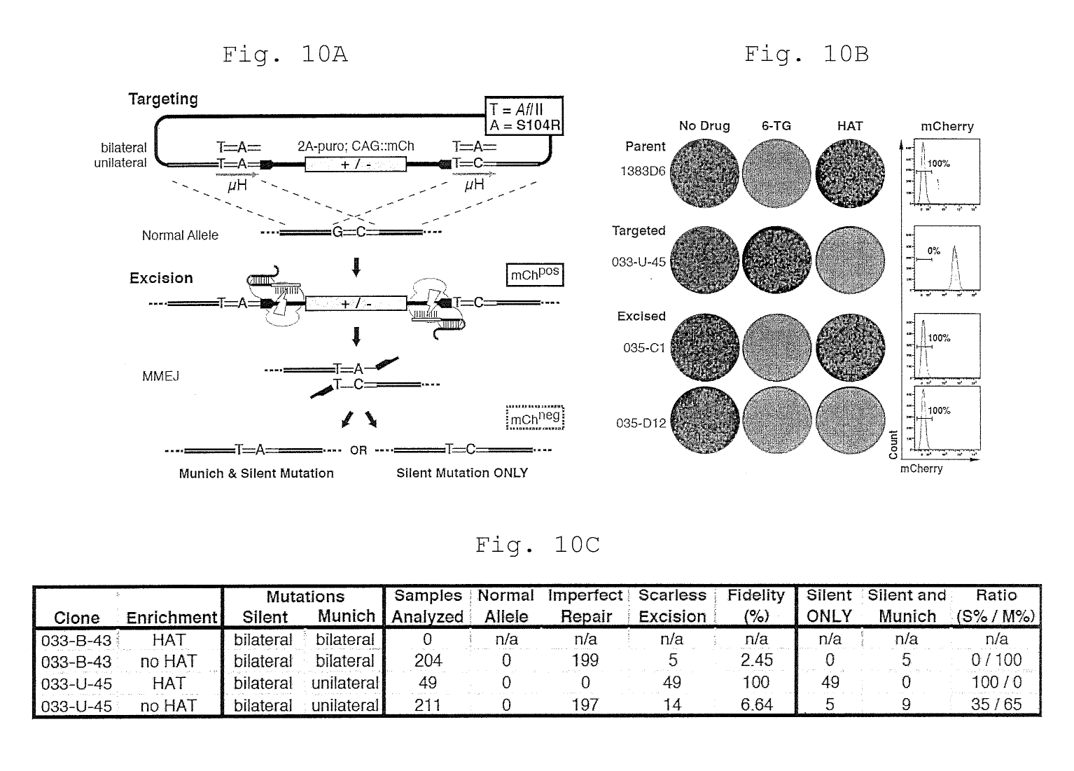

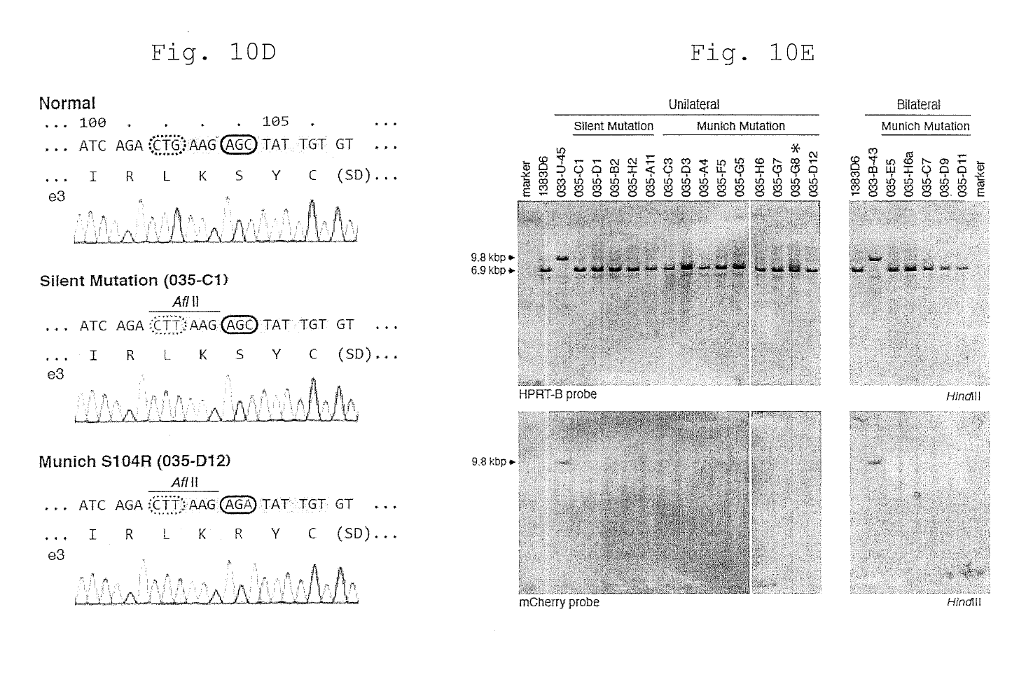

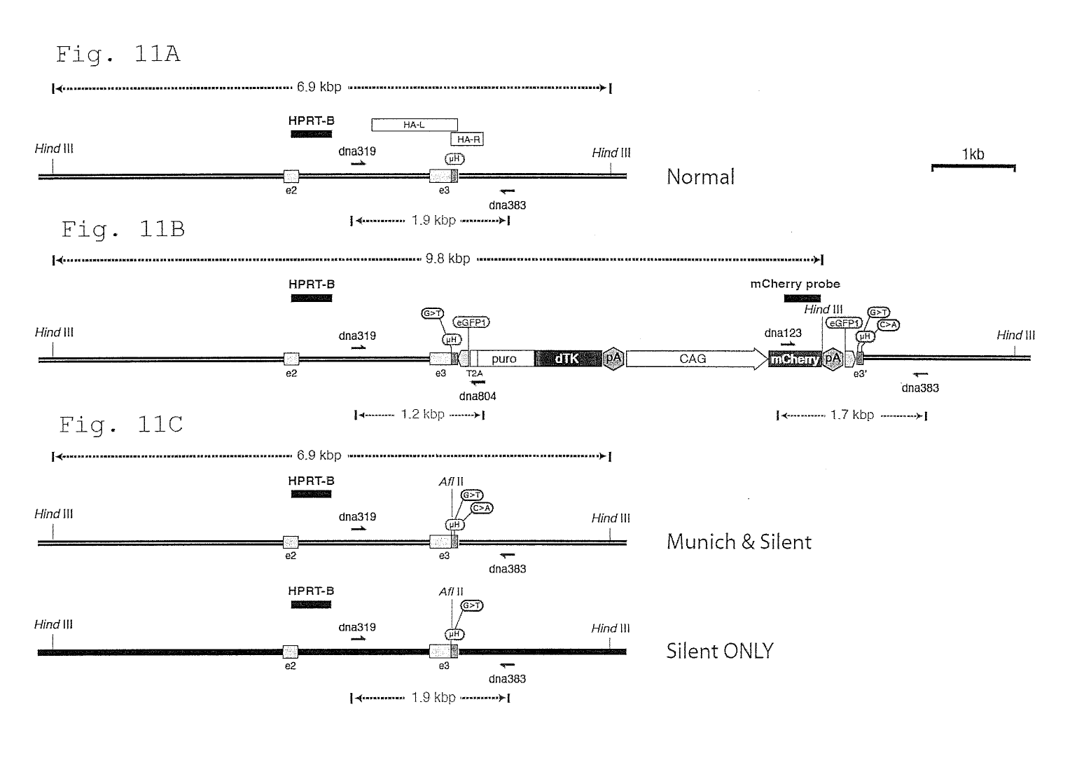

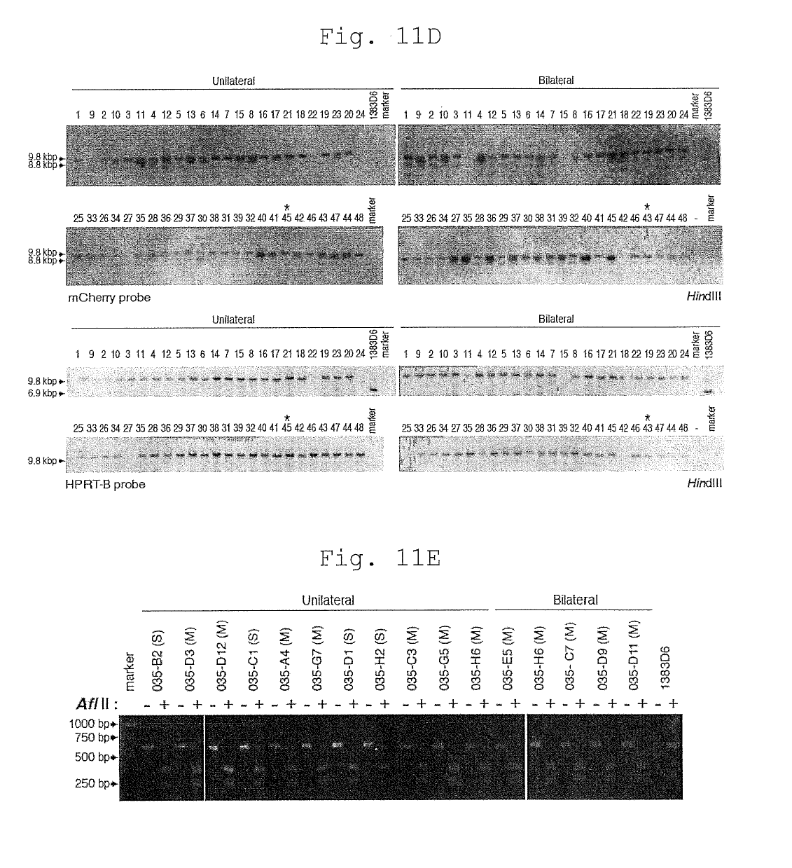

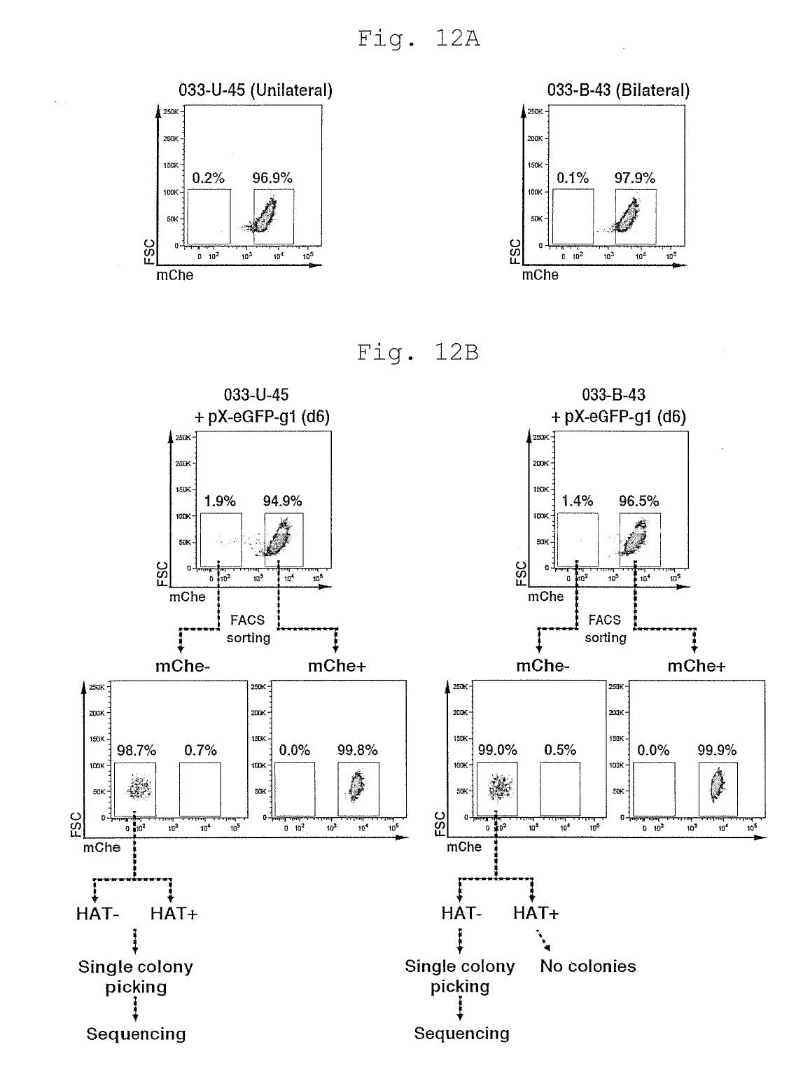

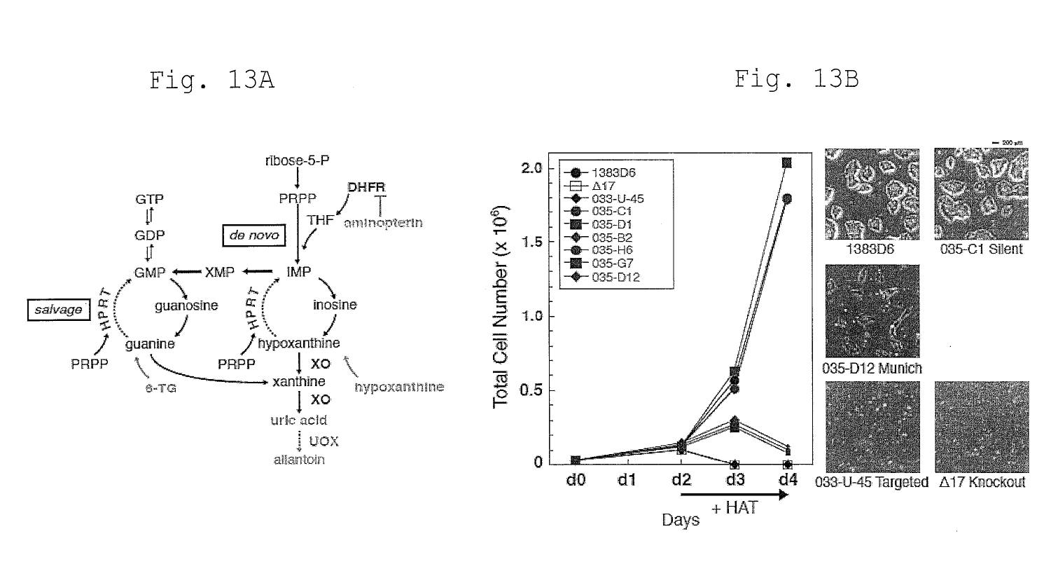

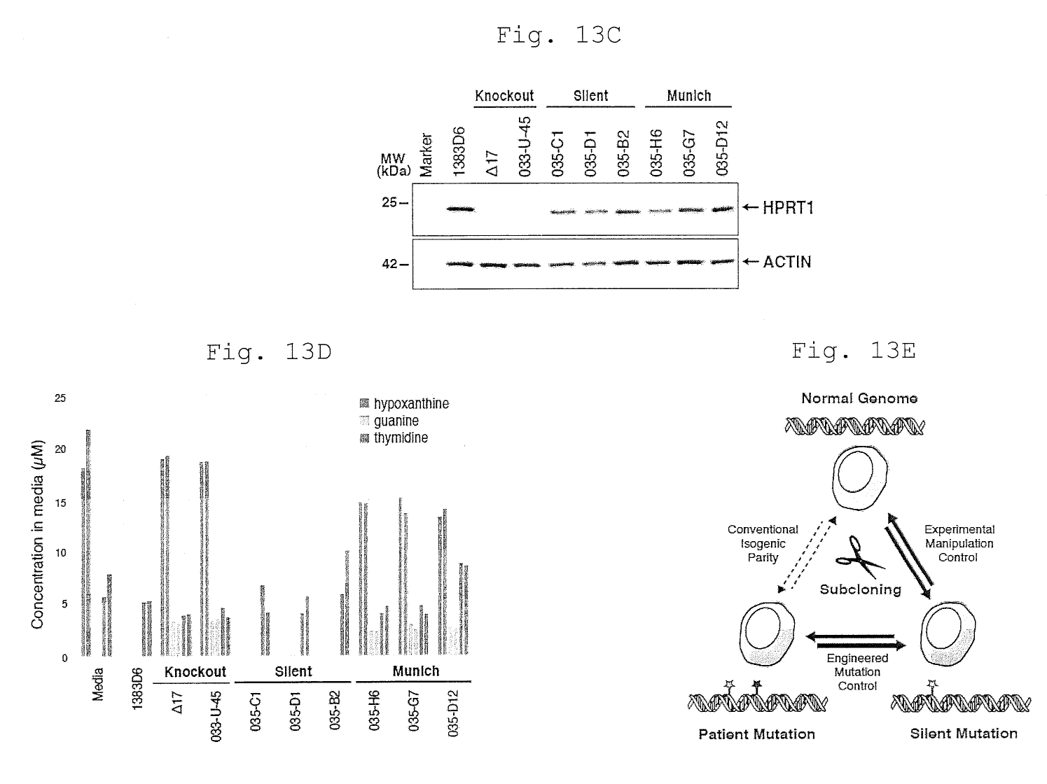

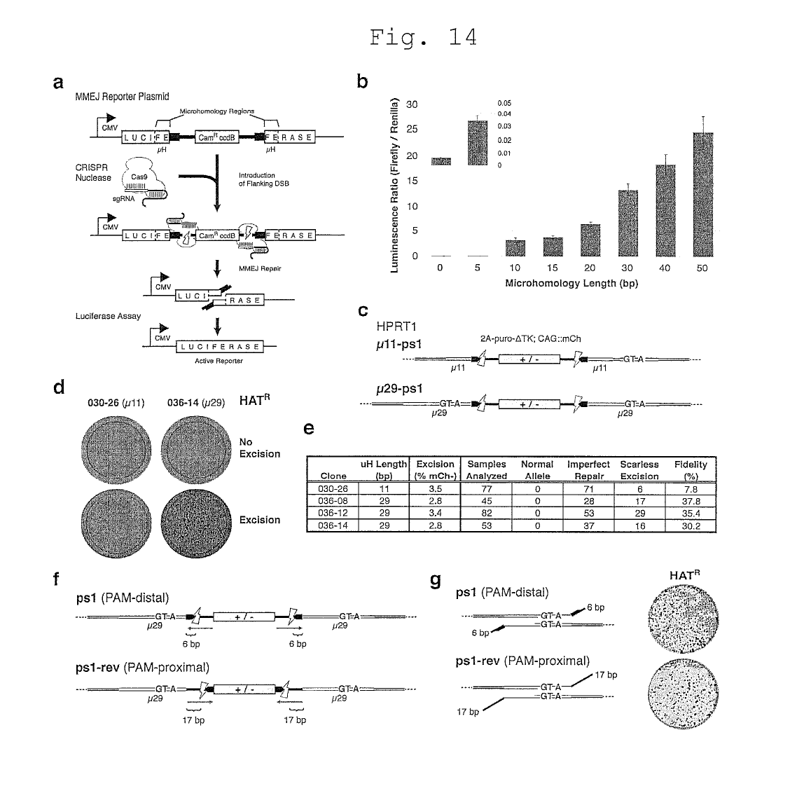

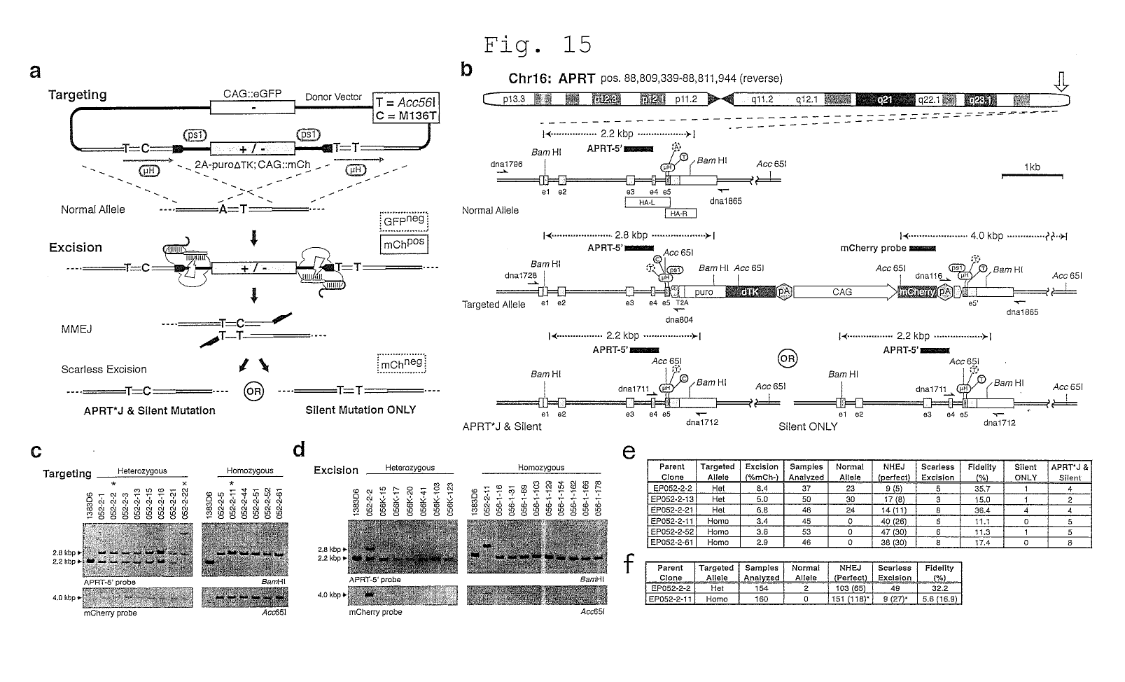

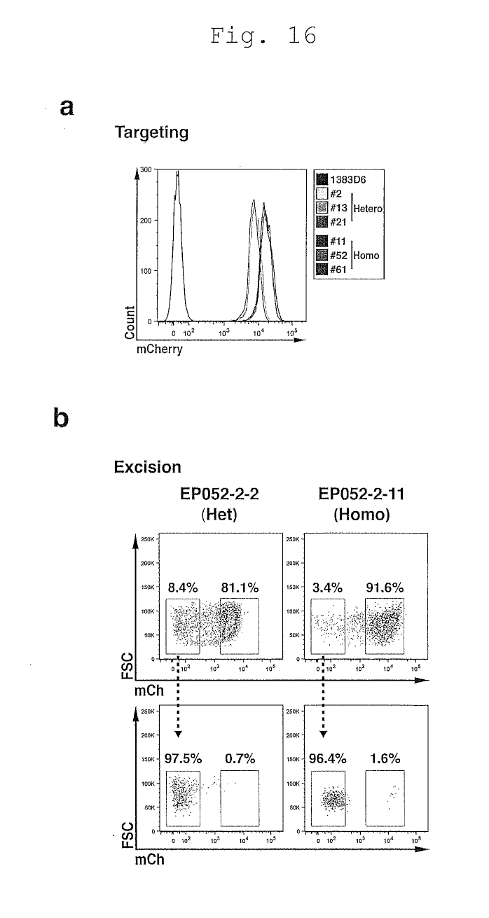

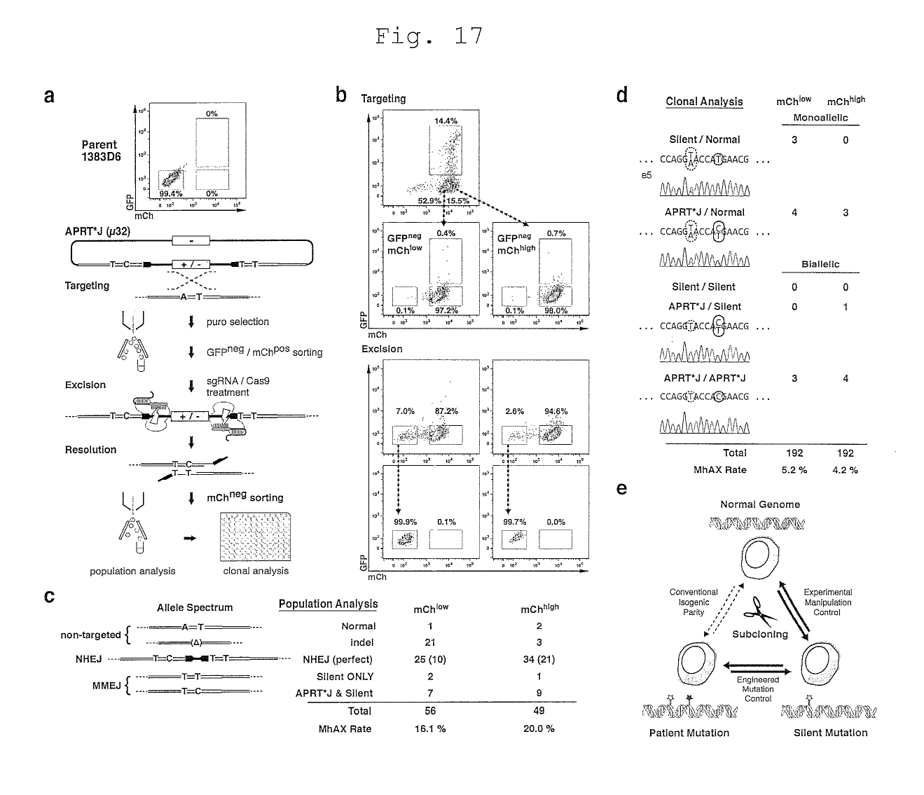

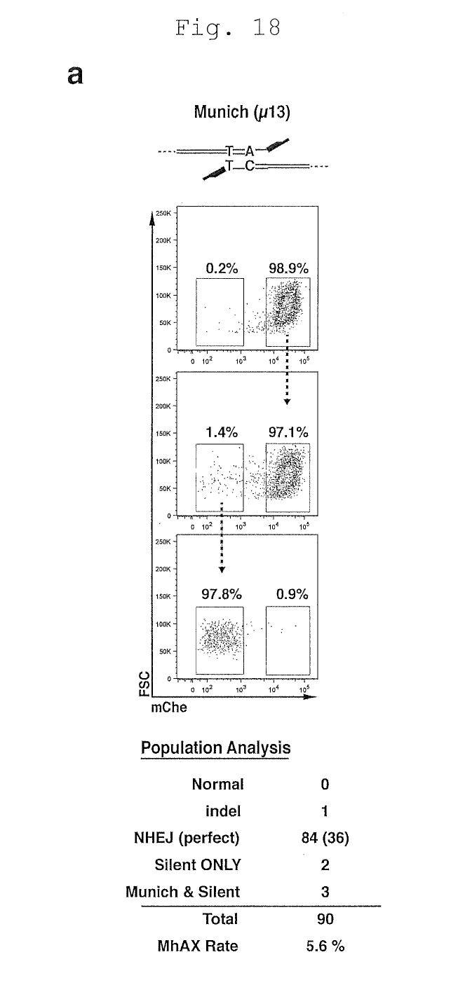

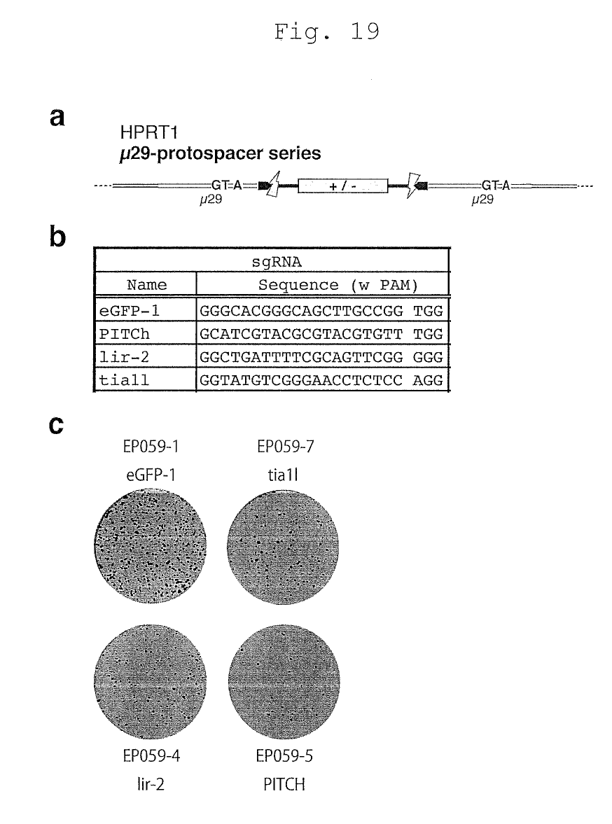

Sequence of HPRT1 alleles from 409B2 (female) iPSC clones treated with HPRT1_B NC-TALENs and enriched by 6-TG selection on SNL feeders. Under SNL feeder conditions, many female iPSCs have two active X-chromosomes (Tomoda et al., Cell stem cell 11, 91-99, 2012), and therefore require disruption of both HPRT1 alleles to resist 6-TG selection (Sakuma et al., Genes Cells 18,315-326, 2013). PCR amplicons of the target site were TA-cloned and at least 8 bacterial colonies from each transformation were PCR-amplified to determine individual alleles by Sanger sequencing. Clones are labeled numerically and alleles alphabetically. iPSC clones with more than two alleles likely represent mosaic populations. Upper case letters represent TALEN binding sites (FIG. 1). Inserted bases are in italics. Deletion or insertion sizes are indicated on the right. REF, parental 409B2 iPSC reference genomic sequence; NORM, non-mutant allele for the region examined by sequencing.

FIG. 3 shows that updated TALEN architecture improves HPRT1_B cleavage activity.

A. SSA Assay comparing the activity of HPRT1_B TALENs assembled using a Xanthomonas oryzae pv. (PthXo1)-based TALE scaffold (NC-TALEN, Sakuma et al., Genes Cells 18, 315-326, 2013), or improved X. campestris pv. vesicatoria (AvrBs3)-based +136/+63 scaffold (Avr-TALEN, Sakuma et al., Scientific reports 3, 3379, 2013). PthXo1-based AAVS1 NC-TALENs (Oceguera-Yanez et al., Methods 101, 43-55, 2016) are included as a reference. Ratio, calculated values for the ratio of measured Firefly/Renilla luciferase activity.

B. TALEN activity in 1383D6 male iPS cells as measured by 6-TG^{R} colony formation, indicating HPRT1 disruption. Spontaneous colony formation in the absence of nuclease was not noted. For the assay, 1 μg of each nuclease was transfected into 1×10⁶ cells by electroporation, followed by plating at a density of 5×10⁵ cells per 60 mm dish. iPSCs were selected and stained as described in the Materials and Methods.

C. Avr-TALENs achieve higher levels of gene targeting in 1383D6 iPSCs as determined by puro^{R} colony formation upon co-transfection with a positive-selection donor plasmid (FIG. 7A). An in-frame gene trap is required to activate the promoterless 2A-puro cassette, and therefore off target insertion or random integration is rare. Spontaneous colony formation in the absence of nuclease was not noted (not shown). For the assay, 1 μg of each nuclease and 3 μg of donor vector were transfected into 1×10⁶ cells by electroporation, followed by plating at a density of 5×10⁵ cells per 60 mm dish. iPSCs were selected and stained as described in the Materials and Methods.

FIG. 4 shows TIDE analysis of indel formation at the HPRT1_B TALEN target site.

A. Schematic of the genomic PCR assay used to analyze the locus targeted by HPRT1_B TALENs. For TIDE analysis, the breakpoint was positioned at the beginning of the spacer as indicated (black arrow).

B. Sequence trace files of the original 1383D6 iPSCs, and 6-TG^{R} population following treatment with TALENs. The position of the breakpoint used for TIDE analysis is shown (black arrow). An ambiguous A/T base is noted upstream of the predicted breakpoint (red arrow).

C. Aberrant sequence plot determined by the online TIDE software. Arrows are as in B.

D. Spectrum of indels in the mixed 6-TG^{R} iPSC population as predicted by TIDE. Deletions are more common than insertions, with a clear bias towards 17 bp deletions. The data in Panel C and D was reproduced across independent experiments (n=3).

E. Sequence trace files of the original H1 ESCs, and 6-TG^{R} population following treatment with TALENs. The position of the breakpoint used for TIDE analysis is shown (black arrow). An ambiguous base is noted upstream of the predicted breakpoint (red arrow).

F. Aberrant sequence plot determined by the online TIDE software. Arrows are as in E.

G. Spectrum of indels in the mixed 6-TG^{R} ESC population as predicted by TIDE. As with 1383D6 iPSCs, deletions are more common than insertions, with a clear bias towards 17 bp deletions.

FIG. 5 shows spectrum of Avr-TALEN-induced mutations in human male iPSCs clones.

Sequence of HPRT1 alleles types detected in a series of individual clones derived from 1383D6 (male) iPSC clones treated with HPRT1_B Avr-TALENs and enriched by 6-TG selection under feeder-free conditions. PCR amplicons of the target site were directly Sanger sequenced. Clones are labeled numerically. Mixed sequences were not included in the analysis. Upper case letters represent HPRT1_B Avr-TALEN binding sites. Inserted bases are in italics. Deletion or insertion sizes are indicated on the right. Of the 4 complex alleles indicated in FIG. 1C, three were delta17T alleles with additional missense mutations or inserted bases (samples not shown). Apart from delta17 the most common deletion was delta46 (10% or 3/30 deletions), where the deletion boundaries were positioned within T-rich sequences following a predicted ‘GATT’ μH. REF, parental 1383D6 iPSC reference genomic sequence.

FIG. 6 shows drug sensitivities of 1383D6 parental and HPRT1 knockout iPSC clones.

Crystal violet staining of representative HPRT1 knockout clonal iPSC lines following treatment with 6-TG or HAT media for 3 days. Resistance and sensitivity correlates with the status of the HPRT1 locus, as determined by PCR genotyping and sequencing (FIG. 5).

FIG. 7 shows that engineered microhomology enables seamless cassette excision to deposit point mutations.

A. Schematic of the MhAX technique used to silently modify the HPRT locus. The donor vector homology arms are engineered with overlap to generate 11 bp tandem microhomology (μH; blue) flanking the positive/negative (+/−) antibiotic selection cassette (grey). Complementary protospacer sequences (black) are nested between the μH and cassette in a divergent orientation. The protospacer sequence and positions of the cut site are indicated above (green). In this example, endogenous μ5T3 (FIG. 1A) was employed in the μH, and mutations (red) are positioned in the unique region of the right homology arm, disrupting the endogenous μ5A3 sequence. HPRT1_B Avr-TALENs (not shown) are used to enhance gene targeting, and positive selection with puromycin enriches for targeted clones. Upon treatment with CRISPR/Cas9, flanking DSBs are generated proximal to the engineered μH. Repair by MMEJ scarlessly excises the cassette, leaving behind only the three silent mutations (red). Gene targeting and screening are detailed in FIG. 3.

B. Reversal of drug resistance during engineering of the HPRT1 locus as shown by crystal violet staining of iPSC colonies. Resistance to puromycin (puro) indicates the presence of the targeting cassette, while 6-TG and HAT resistance indicate HPRT enzymatic deficiency or activity, respectively. The engineered mutations shown in Panel A are silent, as intended.

C. Southern blot analysis of HAT-selected clones reveals restoration of the HPRT1 locus (HPRT-B probe, left) without detectable re-integration of the cassette (TK probe, right). Original 1383D6 and parental 016-A3 targeted iPSC clones are included as controls.

D. MMEJ rates and excision fidelity were determined with or without HAT selective pressure. Only high quality sequence reads were considered in the analysis. MMEJ Rate is calculated as (MMEJ Repair/Samples Analyzed). Scarless excision refers to MMEJ repair events without any additional base mutations. ‘Fidelity’ is calculated as (‘Scarless Excision’/‘MMEJ Repair’).

E. Sequence trace file of an iPSC clone following cassette excision via scarless MMEJ (left) or classic NHEJ (right), the latter resulting from direct fusion of the ends predicted to be formed by CRISPR-induced DSBs.

FIG. 8 shows targeting the HPRT locus with excisable cassettes to deposit silent point mutations.

A. Schematic showing part of the normal HPRT allele. Exons are shown in grey. Overlapping homology arms (HA-L/R) are shown in white. The μH region is shown in blue. Black bars indicate Southern blot probes. Primers used for screening targeted clones are shown in red.

B. Schematic of the targeted HPRT allele, including details on PCR and Southern blot screening strategies. The promoterless 2A-puro-deltaTK cassette is inserted in-frame with HPRT exon 3. CRISPR target sites for eGFP1 are shown in green. Silent mutations are highlighted in red.

C. Schematic of the excised HPRT allele, with deposited mutations.

D. Sanger sequencing results for clone 016-A3 showing the junctions of the locus and cassette (grey) after targeting. The flanking μH (blue), eGFP1 protospacers (green) with predicted cleavage sites (green arrows), and silent point mutations (red) are shown.

E. Southern blotting results for select clones following gene-targeting. The predicted band sizes shown in Panel A and B are indicated. 1383D6 iPSCs are included as a control.

F. Crystal violet staining of HAT^{R} colony formation from 016-A3 iPSCs treated with the pX330-based eGFP1 sgRNA expression vector, indicating cassette excision and restoration of the HPRT locus. HATR colonies were not observed in the absence of nuclease or following transfection of a pX330 vector encoding a non-targeting sgRNA, eGFP2.

FIG. 9 shows Screening sgRNAs for cleavage activity.

A. Diagram of the pX330 sgRNA and Cas9 expression vector (Ran et al., 2013), and the associated pGL4-SSA target plasmids used for the plasmid cleavage assay. The three eGFP protospacer sequences (Fu et al., 2013b) are shown.

B. Relative SSA activities as determined by luciferase expression.

C. A transgene disruption assay was designed to assess genomic cleavage activity in iPSCs. 317-A4 iPSCs are heterozygous for a constitutively expressed CAG::eGFP reporter transgene targeted to the AAVS1 locus (Oceguera-Yanez et al., Methods 101, 43-55, 2016). Relative positions of the three sgRNAs is shown. Microscopy and FACS analysis for GFP expression 6 days after nuclease treatment was used to compare the activities of the three sgRNAs. Scale bar, 200 μm.

FIG. 10 shows that imperfect microhomology simultaneously creates iPSCs with patient mutations and their isogenic controls.

A. Schematic of the MhAX technique to produce the HPRT_{Munich} patient mutation and isogenic control iPSCs. The donor vector and cassette are engineered essentially as described in FIG. 7A, with some key differences. The flanking 13 bp μH is positioned with the S104 codon centrally, and modified with the patient mutation (C>A) or only one side (unilateral) or on both sides (bilateral). A silent point mutation (G>T) generating a diagnostic AflII restriction site is included bilaterally. The positive/negative selection cassette employs a constitutive CAG::mCherry reporter to monitor targeting and excision steps. HPRT1_B Avr-TALENs (not shown) are used to enhance gene targeting, and positive selection with puromycin and mCherry enriches for targeted clones. Upon treatment with CRISPR/Cas9, flanking DSBs are generated proximal to the engineered μH. Repair by MMEJ scarlessly excises the cassette, resulting in two possible outcomes of engineered mutations. Excised clones are mCherry negative.

B. Reversal of 6-TG and HAT drug sensitivities during engineering of the HPRT1 locus as shown by crystal violet staining of iPSC colonies only occurs for clones with a silent mutation (035-C1), while clone 035-D12 remains sensitive to both drugs. Original 1383D6 and unilateral parent clone 033-U-45 are included as controls. FACS analysis for mCherry is shown on the right.

C. MMEJ rates and excision fidelity were determined for clones with unilateral or bilateral mutations, with or without HAT selective pressure. Calculations are as in FIG. 7D.

D. Sequence trace files of iPSC clones with silent only or Munich mutations following scarless MMEJ cassette excision from clone 033-U-45 (unilateral mutations). Both types of clones were isolated from the same experiment.

E. Southern blot analysis of excised clones reveals restoration of the HPRT1 locus (HPRT-B probe, top) without detectable re-integration of the cassette (mCherry probe, bottom). Original 1383D6 and parental 033-U-45 and 033-B-43 targeted iPSCs are included as controls. An asterisk (*) indicates the detection of a secondary band in clone 035-G8, and drug selection confirmed mosaicism (data not shown).

FIG. 11 shows Targeting the HPRT locus with MhAX selection markers bearing imperfect microhomology.

A. Schematic showing part of the normal HPRT allele. Exons are shown in grey. Overlapping homology arms (HA-L/R) are shown in white. The μH region is shown in blue. Black bars indicate Southern blot probes. Primers used for screening targeted clones are shown in red.

B. Schematic of the targeted HPRT allele, including details on PCR and Southern blot screening strategies. The promoterless 2A-puro-deltaTK; CAG::mCherry selection marker is inserted in-frame with HPRT exon 3. CAG::mCherry improves detection of the targeting and excision. CRISPR target sites for eGFP1 are shown in green. Silent mutations are highlighted in red.

C. Schematic of the two potential HPRT alleles following excision, with either Silent and Munich (top) or only Silent (bottom) mutations deposited. The AflII site generated by the Silent mutation is indicated.

D. Southern blotting results for 96 iPSC clones each targeted with either unilaterally or bilaterally mutant μH, and probed with either mCherry (top) or HPRT (bottom). The predicted 6.8 kbp (normal) and 9.8 kbp (targeted) band sizes shown in Panels A and

B are indicated, along with an 8.8 kbp band which arises as a result of donor vector backbone integration, the most common source of background when using a circular plasmid donor with gene-trap selection (Oceguera et al.). Selected clones (033-U-45 and 033-B-43) are indicated with an asterisk. 1383D6 iPSCs are included as a control.

E. AflII digestion of PCR amplicons following MhAX from iPSC clones engineered with unilateral or bilateral homology, indicating the presence of the Silent (S) mutation in all clones tested. Clones labelled with ‘M’ were found to also contain the Munich mutation by sequencing. 1383D6 iPSCs are included as a negative control for cleavage.

FIG. 12 shows isolation of cassette-excised clones by FACS.

A. Outline of FAGS sorting scheme used to enrich cassette-excised clones 6 days after treatment with the eGFP1 sgRNA expression vector. Similar excision rates (˜1-2%) were observed amongst multiple clones with either bilateral or unilateral μH.

B. mCherry-negative and-positive cell populations were sorted and verified for purity, then plated with or without HAT selection. Clonal analysis was performed to determine the frequency and fidelity of MhAX, and the ratios of point-mutation deposition for unilateral μH. The results are summarized in FIG. 10E. Based on the observed rate of repair of μ11 in the absence of selective pressure (˜15%), we chose to plate cells under HAT selection at a 10-fold higher density than unselected in order to obtain similar colony numbers.

FIG. 13 shows that Metabolic phenotyping confirms purine salvage defects in HPRT_{Munich} iPSCs.

A. De novo synthesis and salvage pathways in purine metabolism. HPRT catalyzes both the conversion of guanine to guanine monophosphate (GMP), and hypoxanthine to inosine monophosphate (IMP). With complete or partial HPRT deficiency, metabolites accumulate. Xanthine oxidase (XO) converts hypoxanthine into uric acid. Unlike most mammals, humans lack uric acid oxidase (UOX) and do not enzymatically convert uric acid into allantoin.

B. Growth curve analysis of parental and engineered iPSCs in the presence of HAT selective pressure. HPRT_{Munich} iPSCs show a reduced sensitivity to HAT compared to knockouts (delta17) or targeted parental clone 033-U-45. The growth of iPSCs with Silent mutations are indistinguishable from 1383D6. Note that the behavior of individual clones with similarly engineered genotypes were comparable. Representative morphology of iPSCs colonies after 24 hrs of HAT selection is shown on the right. Scale bar, 200 μm.

C. Western blot analysis of HPRT protein levels in parental and engineered iPSC clones. Knockout lines delta17 and 033-U-45 produce no HPRT protein. Expression levels in HPRT_{Munich} and Silent control clones are comparable to normal 1383D6 iPSCs. ACTIN is used as a loading control.

D. CE-MS metabolite assay of spent media from parental and engineered iPSCs. Hypoxanthine and guanine accumulate as a result of HPRT deficiency, with a less severe phenotype in HPRT_{Munich} cells. Silent control iPSCs behave similarly to 1383D6. Thymidine levels remain essentially unchanged. Data from two independent samples is shown (n=2).

E. The creation of isogenic controls from patient or normal iPSCs is facilitated by genome engineering. Conventional controls for engineered cells (bottom left) come directly from the parent iPSCs (top), yet extended passage and genetic manipulation methods impose sources of technical variation that cannot be accounted for. Using MhAX with imperfect microH, isogenic controls which have undergone comparable experimental manipulations (bottom right) may be isolated simultaneously, providing a new dimension to the interdependence of isogenic controls.

FIG. 14 shows parameters affecting MMEJ fidelity.

a. Schematic of the plasmid-based MMEJ assay mimicking excision from the iPSC chromosome. MMEJ efficiency is measured via luciferase activation. Bacterial selection markers allow for plasmid recovery and genotyping of repair events.

b. MMEJ assay result showing a correlation between luciferase activity and increasing length of flanking microhomology. Inset shows low-level luciferase activity with 5 bp microH compared to background.

c. Schematic of MhAX cassettes with 11 or 29 bp of microH targeted to the HPRT locus.

d. HAT resistant colonies following excision of the cassettes shown in c.

e. Genotyping results from excised clones showing higher MMEJ rates with longer homology.

f. Inversion of the flanking protospacers to examine the role of heterology on MMEJ repair rates.

g. HAT resistant colonies following excision of the cassettes shown in f.

FIG. 15 shows that imperfect microhomology simultaneously creates iPSCs with patient mutations and their isogenic controls.

a. Schematic of the MhAX technique with unilateral microH to produce the APRT*J patient mutation and isogenic control iPSCs. A GFP reporter is included in the backbone to exclude random integration.

b. Genotyping of APRT gene targeting intermediates and final clones.

c. Southern blotting results for APRT gene targeting.

d. Southern blotting results for APRT cassette excision.

e. Summary of genotyping data following MhAX excision showing the APRT allele spectrum (clones).

f. Summary of diploid genotypes of all clonally isolated iPSCs

FIG. 16 shows flow cytometry analysis of APRT gene targeting and excision.

a. Histograms of mCherry fluorescence in targeted clones.

b. FACS plots showing sorting of mCherry-negative cells following MhAX excison.

FIG. 17 shows expedited APRT gene editing using FACS sorting.

a. Schematic of the FACS sorting protocol to isolate targeted and excised iPSCs.

b. FACS plots for APRT gene editing.

c. Allele spectrum and distribution within the excised population.

d. Allele spectrum and distribution amongst excised clones.

e. A novel source of isogenically paired iPSC clones.

FIG. 18 shows expedited HPRT gene editing using FACS sorting.

FIG. 19 shows alternate protospacer use for MhAX.

a. Schematic of MhAX cassettes with 29 bp of microH and various flanking protospacers targeted to the HPRT locus.

b. List of protospacers tested in the HPRT repair assay.

c. HAT-resistant colonies arising from cassette excision and MMEJ repair.

### DESCRIPTION OF EMBODIMENTS

The present invention provides a method of producing a cell having a scarless genome sequence wherein an exogenous nucleic acid sequence inserted into a targeted region in the genome is completely excised (hereinafter also referred to as “the method of the present invention”).

Herein, the term “scarless” means that a targeted region of a genome sequence into which an exogenous nucleic acid sequence has been inserted is restored to its former state without residual fragment of the exogenous nucleic acid sequence and deletion of endogenous genome sequence.

Herein, the term “targeted region” means a site in the genome into which the exogenous nucleic acid sequence is inserted and the vicinity thereof, which can be arbitrarily chosen from the entire region of the genome of host cell. In an embodiment, the targeted region may be a region containing a site where a mutation is to be introduced (or a mutation is to be restored) in the genome sequence.

### 1. Exogenous Nucleic Acid Sequence

The “exogenous nucleic acid sequence” to be removed from the genome sequence in the present invention comprises:

(a) a nucleic acid sequence homologous to a genome sequence in the targeted region at each end (hereinafter also referred to as “homologous nucleic acid sequence”), and

(b) one or more sequence-specific nuclease-recognizing site(s) between the two homologous nucleic acid sequences.

### «Homologous Nucleic Acid Sequence»

The homologous nucleic acid sequence of the aforementioned (a) is not limited, as long as DNA repair by microhomology-mediated end joining (MMEJ) or single-strand annealing occurs between two cleaved ends containing the homologous nucleic acid sequences that have been generated by double-strand break (DSB) at the sequence-specific nuclease-recognizing site(s) of the aforementioned (b). As an Example of the homologous nucleic acid sequence, a sequence homologous to a nucleic acid sequence consisting of contiguous about 5 to 1,000 nucleotides located in the targeted region is included. It is said that, in nature, MMEJ occurs mediated by microhomology sequences consisting of about 5 to 25 nucleotides, whereas SSA occurs mediated by longer homologous sequences (e.g., not less than 30 nucleotides). However, in the present invention, since both end-repair mechanisms result in the same outcome, it is not important to precisely determine which mechanism is utilized. However, considering easiness of construction of the homologous nucleic acid sequence of the present invention and the like, the nucleotide length of the homologous nucleic acid sequence is preferably 5 to 100 nucleotides or 5 to 50 nucleotides. It is known that repair efficiency by MMEJ is improved, as the length of microhomology sequence increases (Villarreal et al., 2012). In fact, the present inventors confirmed that repair efficiency is improved in sequence length-dependent manner, at least within the range of 5 to 50 nucleotides, in preliminary studies using plasmid end joining assay.

Herein, the term “homologous” encompasses not only when two nucleic acid sequences are completely the same but also when one to several (e.g., 1, 2 or 3) nucleotides are different between the sequences. Therefore, the homologous nucleic acid sequence contained in the exogenous nucleic acid sequence can have one to several mutations against the corresponding endogenous genome sequence. Also, the two homologous nucleic acid sequences may be completely the same, or different in one to several nucleotides.

### «Sequence-Specific Nuclease-Recognizing Site»

In the aforementioned (b), the term “sequence-specific nuclease” means a nuclease capable of specifically recognizing a certain target nucleotide sequence and cleaving a double-stranded DNA within the target nucleotide sequence or in the vicinity thereof. The sequence-specific nuclease may be a nuclease having a sequence-specificity per se such as restriction enzymes, or a complex of (i) a molecule or molecule complex (hereinafter also referred to as “nucleic acid sequence recognition module”) having an ability to specifically recognize and bind to a particular nucleotide sequence (i.e., target nucleotide sequence) on a DNA strand, and (i) a non-specific nuclease (e.g., Fok I and the like) linked to the aforementioned (i), wherein the “complex” encompasses not only those consisting of multiple molecules but also those having the nucleic acid sequence recognition module and the nuclease in a single molecule such as a fused protein. The latter is more preferable in that it can confer a recognition capability against a nucleotide sequence longer than a restriction enzyme recognition site to the nuclease. To be specific, as preferable examples of the sequence-specific nuclease are included Zinc-finger nuclease (ZFN), transcription activator-like effector nuclease (TALEN) or clustered regulatory interspaced short palindromic repeats/CRISPR-associated protein (CRISPR/Cas) and the like. In addition, a non-specific nuclease linked to a fragment that contains a DNA-binding domain of a protein capable of specifically binding to DNA such as restriction enzyme, transcription factor, RNA polymerase and the like, but does not have an ability to cleave a double stranded DNA, can also be used as a sequence-specific nuclease. Furthermore, an artificial nuclease in which a PPR protein designed so as to have a sequence specificity by sequential PPR motifs is ligated with a non-specific nuclease can also be used (see JP 2013-128413 A).

The term “sequence-specific nuclease-recognizing site” means a nucleotide sequence that is specifically recognized by any of the aforementioned sequence-specific nucleases, and may include various restriction enzyme recognition sites and cis sequences capable of specifically binding to DNA-binding proteins such as transcription factors, RNA polymerases and the like. However, since they have disadvantages that available nucleotide sequences are limited, and it is highly probable that the target nucleotide sequence (i.e., off-target site) exists in a region other than the targeted region on the genome, preferably, a nucleotide sequence recognized by an artificial nuclease such as ZFN, TALEN, CRISPR/Cas or the like, which has a high degree of freedom for sequence, can be selected as the sequence-specific nuclease-recognizing site.

Since the sequence-specific nuclease-recognizing site is excised from genome sequence upon DNA repair by MMEJ or SSA, any nucleotide sequence can be used as the recognizing site irrespective of the genome sequence in the targeted region. Usually, ZFN or TALEN needs to newly design according to the target nucleotide sequence of interest, but, in the present invention, a nucleotide sequence recognized by existing ZFN or TALEN can be diverted as the sequence-specific nuclease-recognizing site:

One or more sequence-specific nuclease-recognizing sites are located between the two homologous nucleic acid sequences. As long as a repair by MMEJ or SSA occurs between the two homologous nucleic acid sequences generated by DSB at the sequence-specific nuclease-recognizing site, the number of the sequence-specific nuclease-recognizing site may be one. However, in a preferable embodiment, since the exogenous nucleic acid sequence contains one or more exogenous genes (e.g., selectable marker genes such as drug-resistant genes and reporter genes including fluorescent protein genes, and the like), in such case, MMEJ or SSA may not efficiently occur by a single site cleavage. As such, when the exogenous nucleic acid sequence contains a long insertion sequence such as a gene expression cassette between the aforementioned homologous sequences, it is more preferable that the insertion sequence is flanked by two sequence-specific nuclease-recognizing sites. Since the long insertion sequence is deleted by two-site DSBs, two cleaved ends containing the homologous sequences near the ends are generated, which allow DNA repair by MMEJ or SSA.

In this connection, while it is not excluded that an extra nucleotide sequence is added between the homologous nucleic acid sequence and the sequence-specific nuclease-recognizing site, the added nucleotide sequence desirably has a length such that it does not prevent MMEJ or SSA by the two homologous nucleic acid sequences. Therefore, in a preferable embodiment, the homologous nucleic acid sequence substantially lies adjacent to the sequence-specific nuclease-recognizing site.

On the other hand, when the nucleotide sequence inserted between the homologous nucleic acid sequences is sufficiently short, as long as the exogenous nucleic acid sequence contains only one sequence-specific nuclease-recognizing site between the homologous sequences, MMEJ or SSA may occur between the cleaved ends generated by DSB at the site. For example, a target gene on the host genome can be temporarily destructed by inserting the exogenous nucleic acid sequence, and at a desired time, the destructed endogenous gene can be restored by DSB at the sequence-specific nuclease-recognizing site and the subsequent repair by MMEJ or SSA.

Meanwhile, As long as one or two sequence-specific nuclease-recognizing site(s) is/are located such that DSB(s) at the sequence-specific nuclease-recognizing site(s) results in generation of two cleaved ends that may cause repair by MMEJ or SSA, the exogenous nucleic acid sequence may further contain one or more extra sequence-specific nuclease-recognizing sites.

When the exogenous nucleic acid sequence has two or more sequence-specific nuclease-recognizing sites, they may have the same or different nucleotide sequences, but the former is advantageous, considering only one kind of sequence-specific nuclease is required.

### 2. The Method of the Present Invention

The method of the present invention comprises the following steps:

(1) a step of introducing the sequence-specific nuclease or a nucleic acid encoding the same into a host cell having a genome sequence into which the exogenous nucleic acid sequence is inserted; and

(2) culturing the cell obtained in step (1).

The host cell used in the method of the invention is not particularly limited, as long as it is derived from an organism that can be genetically manipulated. Namely, the method of the present invention is applicable to any cell type (for example, somatic cells, somatic stem cells, pluripotent stem cells (e.g., ES cells, iPS cells and the like), and the like) of any organism (for example, bacteria such as Escherichia coli, Bacillus subtilis and the like, yeasts, insects, vertebrates (for example, fishes, amphibia, reptiles, birds, mammals (e.g., human, mouse, rat and the like), plants and the like). In a preferable embodiment, the host cell can be a cell originated from human or other mammals, for example, a pluripotent cell such as ES cell, iPS cell and the like. In another preferable embodiment, the host cell can be a pluripotent stem cell established from human that has a disease-specific genetic mutation.

«Host Cell Having a Genome Sequence into which the Exogenous Nucleic Acid Sequence is Inserted»

The host cell having a genome sequence into which the exogenous nucleic acid sequence used in step (1) is inserted may be prepared by any means, as long as the exogenous nucleic acid sequence is inserted into a targeted region in the genome sequence. In a preferable embodiment, the host cell is a cell prepared by inserting the exogenous nucleic acid sequence into the targeted region in the endogenous genome sequence by homologous recombination. Insertion of the exogenous nucleic acid sequence by homologous recombination is carried out by, for example, introducing a nucleic acid, preferably targeting vector, in which genome sequences adjacent to 5′- and 3′-ends of the host cell genome sequence corresponding to the homologous nucleic acid sequence (hereinafter also referred to as “flanking genome sequences”) are ligated to 5′- and 3′-ends of the exogenous nucleic acid sequence, respectively, into the host cell by a conventional method, and selecting a cell in which the exogenous nucleic acid sequence is inserted into the genome sequence corresponding to the homologous sequence within the targeted region in the genome.

Selection of the homologous recombinant can be performed by, when a selectable marker gene (for example, a gene conferring a resistance to drug such as antibiotic, a reporter gene such as fluorescent protein, and the like) is inserted into the exogenous nucleic acid sequence, using the corresponding selection marker (for example, when the selectable marker gene is a drug-resistant gene, culturing the cell in the presence of the drug). On the other hand, when the exogenous nucleic acid sequence does not contain a selectable marker gene, the homologous recombinant can be selected by, for example, when destruction of an endogenous gene by insertion of the exogenous nucleic acid sequence by homologous recombination results in a change in drug response or auxotrophy, detecting the change.

When preparing the homologous recombinant, one to several (e.g., 2, 3, 4, 5) nucleotide mutations (e.g., substitution, deletion, insertion, addition) can be introduced into the corresponding endogenous genome sequence in the homologous nucleic acid sequences. The mutations can be introduced into either or both of the two homologous nucleic acid sequences. In the latter case, the mutations may be the same or different (e.g., substitution with different nucleotides, mutations at the different sites and the like).

Alternatively, one or more mutations (e.g., substitution, deletion, insertion, addition) can be introduced into the aforementioned flanking genome sequences. The mutations can also be introduced into either or both of the two flanking genome sequences.

In a preferable embodiment, the efficiency of homologous recombination can be improved by introducing, into the host cell, a targeting vector in which sequence-specific nuclease-recognizing sites are inserted into the two flanking genome sequences and a sequence-specific nuclease recognizing the recognition sites. Herein, the sequence-specific nuclease-recognizing sites to be introduced into the flanking genome sequences consist of a nucleotide sequence different from that of the sequence-specific nuclease-recognizing sites contained in the exogenous nucleic acid sequence.

As the sequence-specific nuclease, the below-mentioned sequence-specific nucleases that recognize and cleave the sequence-specific nuclease-recognizing sites contained in the exogenous nucleic acid sequence can also be used. Preferably, artificial nucleases such as ZFN, TALEN, CRISPR/Cas and the like are exemplified.

In another embodiment of the present invention, the host cell having a genome sequence into which the exogenous nucleic acid sequence used in step (1) can be prepared by inserting the exogenous nucleic acid sequence into the targeted region of the endogenous genome sequence using MMEJ. Insertion of the exogenous nucleic acid sequence into the targeted region using MMEJ can be carried out, for example, according to the method described in Nakade et al. (2014). Sine the method does not require the flanking genome sequences, it is advantageous in that a labor for cloning the sequences can be reduced.

### «Step (1) Introduction of Sequence-Specific Nuclease or Nucleic Acid Encoding Same»

The sequence-specific nuclease used in step (1) is a nuclease that can recognize sequence-specific nuclease-recognizing sites contained in the aforementioned exogenous nucleic acid sequence and cleave a double-stranded genome sequence within the recognition sites or in the vicinity thereof. While the above-mentioned sequence-specific nucleases can be used herein, an artificial nuclease (complex of nucleic acid sequence recognition module and nuclease) such as ZFN, TALEN, CRISPR/Cas or the like is preferable.

A zinc finger motif is constituted by linkage of 3-6 different Cys2His2 type zinc finger units (1 finger recognizes about 3 bases), and can recognize a target nucleotide sequence of 9-18 bases. A zinc finger motif can be produced by a known method such as Modular assembly method (Nat Biotechnol (2002) 20: 135-141), OPEN method (Mol Cell (2008) 31: 294-301), CoDA method (Nat Methods (2011) 8: 67-69), Escherichia coli one-hybrid method (Nat Biotechnol (2008) 26: 695-701) and the like. JP 4968498 B can be referred to as for the detail of the zinc finger motif production.

A TAL effector has a module repeat structure with about 34 amino acids as a unit, and the 12th and 13th amino acid residues (called RVD) of one module determine the binding stability and base specificity. Since each module is highly independent, TAL effector specific to a target nucleotide sequence can be produced by simply connecting the module. For TAL effector, a production method utilizing an open resource (REAL method (Curr Protoc Mol Biol (2012) Chapter 12: Unit 12.15), FLASH method (Nat Biotechnol (2012) 30: 460-465), and Golden Gate method (Nucleic Acids Res (2011) 39: e82) etc.) have been established, and a TAL effector for a target nucleotide sequence can be designed comparatively conveniently. JP 2013-513389 A can be referred to as for the detail of the production of TAL effector.

Any of the above-mentioned nucleic acid sequence recognition module can be provided as a fusion protein with a nuclease, or a protein binding domain such as SH3 domain, PDZ domain, GK domain, GB domain and the like and a binding partner thereof may be fused with a nucleic acid sequence recognition module and a nuclease, respectively, and provided as a protein complex via an interaction of the domain and a binding partner thereof. Alternatively, a nucleic acid sequence recognition module and a nuclease may be each fused with intein, and they can be linked by ligation after protein synthesis.

The sequence-specific nuclease of the present invention containing a complex (including fusion protein) wherein a nucleic acid sequence recognition module and a nuclease are bonded may be contacted with a genomic DNA by introducing the sequence-specific nuclease protein, but preferably, by introducing a nucleic acid encoding the sequence-specific nuclease into a cell having the genomic DNA.

Therefore, the nucleic acid sequence recognition module and the nuclease are preferably prepared as a nucleic acid encoding a fusion protein thereof, or in a form capable of forming a complex in a host cell after translation into a protein by utilizing a binding domain, intein and the like, or as a nucleic acid encoding each of them. The nucleic acid here may be a DNA or an RNA. When it is a DNA, it is preferably a double stranded DNA, and provided in the form of an expression vector in which the nucleic acid is located under the control of a promoter that is functional in the host cell. When it is an RNA, it is preferably a single strand RNA.

A DNA encoding the nucleic acid sequence recognition module such as zinc finger motif, TAL effector and the like can be obtained by any method mentioned above for each module.

A DNA encoding the nuclease can be cloned by, for example, synthesizing an oligo DNA primer based on the cDNA sequence information thereof, and amplifying by the RT-PCR method using, as a template, the total RNA or mRNA fraction prepared from the nuclease-producing cells.

The cloned DNA may be directly, or after digestion with a restriction enzyme when desired, or after addition of a suitable linker and/or a nuclear localization signal (each oraganelle transfer signal when the object double stranded DNA is mitochondria or chloroplast DNA), ligated with a DNA encoding a nucleic acid sequence recognition module to prepare a DNA encoding a fusion protein. Alternatively, a DNA encoding a nucleic acid sequence recognition module, and a DNA encoding a nuclease may be each fused with a DNA encoding a binding domain or a binding partner thereof, or both DNAs may be fused with a DNA encoding a separation intein, whereby the nucleic acid sequence recognition module and the nuclease are translated in a host cell to form a complex. In these cases, a linker and/or a nuclear localization signal can be linked to a suitable position of one of or both DNAs when desired.

A DNA encoding a nucleic acid sequence recognition module and a DNA encoding a nuclease can be obtained by chemically synthesizing the DNA chain, or by connecting synthesized partly overlapping oligoDNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (http://www.kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence recognition module and/or a nuclease can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as λphage and the like; insect virus vectors such as baculovirus and the like (e.g., BmNPV, AcNPV); animal virus vectors such as retrovirus, vaccinia virus, adenovirus and the like, and the like are used.

As the promoter, any promoter appropriate for a host to be used for gene expression can be used.

For example, when the host is an animal cell, SRα promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like are used. Of these, CMV promoter, SRα promoter and the like are preferable.

When the host is Escherichia coli, trp promoter, lac promoter, recA promoter, λP_{L} promoter, lpp promoter, T7 promoter and the like are preferable.

When the host is genus Bacillus, SPO1 promoter, SPO2 promoter, penP promoter and the like are preferable.

When the host is a yeast, Gal1/10 promoter, PHO5 promoter, PGK promoter, GAP promoter, ADH promoter and the like are preferable.

When the host is an insect cell, polyhedrin promoter, P10 promoter and the like are preferable.

When the host is a plant cell, CaMV35S promoter, CaMV19S promoter, NOS promoter and the like are preferable.

As the expression vector, besides those mentioned above, one containing enhancer, splicing signal, terminator, polyA addition signal, a selection marker such as drug resistance gene, auxotrophic complementary gene and the like, replication origin and the like on demand can be used.

An RNA encoding a nucleic acid sequence recognition module and/or a nuclease can be prepared by, for example, transcription to mRNA in a vitro transcription system known per se by using a vector encoding DNA encoding the above-mentioned nucleic acid sequence recognition module and/or the nuclease as a template.

A complex of a nucleic acid sequence recognition module and a nuclease enzyme can be expressed in a host cell by introducing an expression vector containing a DNA encoding the nucleic acid sequence recognition module and/or the nuclease into the host cell, and culturing the same.

As the host, genus Escherichia, genus Bacillus, yeast, insect cell, insect, animal cell and the like are used.

As the genus Escherichia, Escherichia coli K12.DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], Escherichia coli C600 [Genetics, 39, 440 (1954)] and the like are used.

As the genus Bacillus, Bacillus subtilis MI114 [Gene, 24, 255 (1983)], Bacillus subtilis 207-21 [Journal of Biochemistry, 95, (1984)] and the like are used.

As the yeast, Saccharomyces cerevisiae AH22, AH22R⁻, NA87-11A, DKD-5D, 20B-12, Schizosaccharomyces pombe NCYC1913, NCYC2036, Pichia pastoris KM71 and the like are used.

As the insect cell when the virus is AcNPV, cells of cabbage armyworm larva-derived established line (Spodoptera frugiperda cell; Sf cell), MG1 cells derived from the mid-intestine of Trichoplusia ni, High Five™ cells derived from an egg of Trichoplusia ni, Mamestra brassicae-derived cells, Estigmena acrea-derived cells and the like are used. When the virus is BmNPV, cells of Bombyx mori-derived established line (Bombyx mori N cell; BmN cell) and the like are used as insect cells. As the Sf cell, for example, Sf9 cell (ATCC CRL1711) Sf21 cell [all above, In Vivo, 13, 213-217 (1977)] and the like are used.

As the insect, for example, larva of Bombyx mori, Drosophila, cricket and the like are used [Nature, 315, 592 (1985)].

As the animal cell, cell lines such as monkey COS-7 cell, monkey Vero cell, Chinese hamster ovary (CHO) cell, dhfr gene-deficient CHO cell, mouse L cell, mouse AtT-20 cell, mouse myeloma cell, rat GH3 cell, human FL cell and the like, pluripotent stem cells such as iPS cell, ES cell and the like of human and other mammals, and primary cultured cells prepared from various tissues are used. Furthermore, zebrafish embryo, Xenopus oocyte and the like can also be used.

As the plant cell, suspend cultured cells, callus, protoplast, leaf segment, root segment and the like prepared from various plants (e.g., grain such as rice, wheat, corn and the like, product crops such as tomato, cucumber, egg plant and the like, garden plants such as carnation, Eustoma russellianum and the like, experiment plants such as tobacco, Arabidopsis thaliana and the like, and the like) are used.

All the above-mentioned host cells may be haploid (monoploid), or polyploid (e.g., diploid, triploid, tetraploid and the like).

An expression vector can be introduced by a known method (e.g., lysozyme method, competent method, PEG method, CaCl₂ coprecipitation method, electroporation method, the microinjection method, the particle gun method, lipofection method, Agrobacterium method and the like) according to the kind of the host.

Escherichia coli can be transformed according to the methods described in, for example, Proc. Natl. Acad. Sci. USA, 69, 2110 (1972), Gene, 17, 107 (1982) and the like.

The genus Bacillus can be introduced into a vector according to the methods described in, for example, Molecular & General Genetics, 168, 111 (1979) and the like.

A yeast can be introduced into a vector according to the methods described in, for example, Methods in Enzymology, 194, 182-187 (1991), Proc. Natl. Acad. Sci. USA, 75, 1929 (1978) and the like.

An insect cell and an insect can be introduced into a vector according to the methods described in, for example, Bio/Technology, 6, 47-55 (1988) and the like.

An animal cell can be introduced into a vector according to the methods described in, for example, Cell Engineering additional volume 8, New Cell Engineering Experiment Protocol, 263-267 (1995) (published by Shujunsha), and Virology, 52, 456 (1973).

### «Step (2) Culture of Host Cell and Induction of DSB and MMEJ»

A cell introduced with a vector can be cultured according to a known method according to the kind of the host.

For example, when Escherichia coli or genus Bacillus is cultured, a liquid medium is preferable as a medium to be used for the culture. The medium preferably contains a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is preferably about 5-about 8.

As a medium for culturing Escherichia coli, for example, M9 medium containing glucose, casamino acid [Journal of Experiments in Molecular Genetics, 431-433, Cold Spring Harbor Laboratory, New York 1972] is preferable. Where necessary, for example, agents such as 3β-indolylacrylic acid may be added to the medium to ensure an efficient function of a promoter. Escherichia coli is cultured at generally about 15-about 43° C. Where necessary, aeration and stirring may be performed.

The genus Bacillus is cultured at generally about 30-about 40° C. Where necessary, aeration and stirring may be performed.

Examples of the medium for culturing yeast include Burkholder minimum medium [Proc. Natl. Acad. Sci. USA, 77, 4505 (1980)], SD medium containing 0.5% casamino acid [Proc. Natl. Acad. Sci. USA, 81, 5330 (1984)] and the like. The pH of the medium is preferably about 5-about 8. The culture is performed at generally about 20° C.-about 35° C. Where necessary, aeration and stirring may be performed.

As a medium for culturing an insect cell or insect, for example, Grace's Insect Medium [Nature, 195, 788 (1962)] containing an additive such as inactivated 10% bovine serum and the like as appropriate and the like are used. The pH of the medium is preferably about 6.2-about 6.4. The culture is performed at generally about 27° C. Where necessary, aeration and stirring may be performed.

As a medium for culturing an animal cell, for example, minimum essential medium (MEM) containing about 5-about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium is preferably about 6-about 8. The culture is performed at generally about 30° C.-about 40° C. Where necessary, aeration and stirring may be performed.

As a medium for culturing a plant cell, for example, MS medium, LS medium, B5 medium and the like are used. The pH of the medium is preferably about 5-about 8. The culture is performed at generally about 20° C.-about 30° C. Where necessary, aeration and stirring may be performed.

As mentioned above, a complex of a nucleic acid sequence recognition module and a nuclease, i.e., sequence-specific nuclease, can be expressed within a host cell.

An RNA encoding a nucleic acid sequence recognition module and/or a nuclease can be introduced into a host cell by microinjection method, lipofection method and the like. RNA introduction can be performed once or repeated plural times (e.g., 2-5 times) at suitable intervals.

During the culturing step of step (2), when the sequence-specific nuclease is expressed by an expression vector or RNA molecule introduced into the host cell, the nucleic acid sequence recognition module specifically recognizes and binds to sequence-specific nuclease-recognizing sites in the exogenous nucleic acid sequence inserted into a genome sequence, and DSB occurs within the recognition sites or in the vicinity thereof due to the action of the nuclease linked to the nucleic acid sequence recognition module. Since the resulting cleaved ends contain the homologous nucleic acid sequences, MMEJ or SSA occurs utilizing these sequences, which results in a cell having a scarless genome sequence (i.e., a contiguous sequence consisting of 5′-flanking genome sequence—a single homologous nucleic acid sequence—3′-flanking genome sequence), wherein the exogenous nucleic acid sequence has been completely removed from the targeted region.

In the present invention, since any the sequence-specific nuclease-recognizing site can be used (the same recognition site can be used in any case), it is not necessary to newly design a ZF-motif or TAL-effector for the respective recognition sites (target nucleotide sequences). However, CRISPR-Cas system is more preferable in that any sequence can be targeted by simply synthesizing an oligoDNA capable of specifically hybridizing with the target nucleotide sequence, since CRISPR-Cas system recognizes a double stranded DNA sequence of interest by a guide RNA complementary to the target nucleotide sequence. Therefore, in a preferable embodiment of the present invention, CRISPR/Cas system is used as a sequence-specific nuclease.

The Cas protein to be used in the present invention is not particularly limited as long as it can form a complex with a guide RNA and recognize and bind to a target nucleotide sequence in a gene of interest and a protospacer adjacent motif (PAM) adjacent thereto, but is preferably Cas9 or Cpf1. Examples of Cas9 include, but are not limited to, Streptococcus pyogenes-derived Cas9 (SpCas9; PAM sequence: NGG (N is A, G, T or C. The same shall apply hereinafter.)), Streptococcus thermophiles-derived Cas9 (StCas9; PAM sequence: NNAGAAW), Neisseria meningitidis-derived Cas9 (NmCas9; PAM sequence: NNNNGATT) and the like. While SpCas9 with less constraint of PAM is frequently used, since the target nucleotide sequence can be freely designed in the present invention, Cas9 derived from other species can also be preferably used. On the other hand, Examples of Cpf1 include, but are not limited to, Francisella novicida-derived Cpf1 (FnCpf1; PAM sequence: NTT), Acidaminococcus sp.-derived Cpf1 (AsCpf1; PAM sequence: NTTT), Lachnospiraceae bacterium-derived Cpf1 (LbCpf1; PAM sequence: NTTT) and the like.

Even when CRISPR/Cas is used as a sequence-specific nuclease, it is desirably introduced, in the form of a nucleic acid encoding the same, into a host cell, similar to when ZFN and the like are used as a sequence-specific nuclease.

A DNA encoding Cas can be cloned by a method similar to the above-mentioned method for a DNA encoding a nuclease, from a cell producing the enzyme.

On the other hand, a DNA encoding guide RNA can obtained by designing an oligo DNA sequence linking a DNA sequence complementary to the target nucleotide sequence and a known tracrRNA sequence (e.g., gttttagagctagaaatagcaagttaaaataaggctagtccgttatcaacttgaaaaagtgg caccgagtcggtggtgctttt) and chemically synthesizing using a DNA/RNA synthesizer. While a DNA encoding guide RNA can also be inserted into an expression vector similar to the one mentioned above, according to the host. As the promoter, pol III system promoter (e.g., SNR6, SNR52, SCR1, RPR1, U6, H1 promoter etc.) and terminator (e.g., T₆ sequence) are preferably used.

When CRISPR/Cas is used as a sequence-specific nuclease, the sequence-specific nuclease-recognizing site needs to contain a DNA-cleaving site-recognizing sequence necessary for recognition of DSB site by Cas, PAM (see above regarding the specific PAM sequence), in addition to a nucleotide sequence complementary to crRNA sequence contained in the guide RNA (i.e., target nucleotide sequence).

An RNA encoding Cas can be prepared by, for example, transcription to mRNA, by in vitro transcription system known per se, using a vector carrying a DNA encoding the Cas as a template.

Guide RNA can be obtained by designing an oligo DNA sequence linking a DNA sequence complementary to the target nucleotide sequence and a known tracrRNA sequence and chemically synthesizing using a DNA/RNA synthesizer.

A DNA or RNA encoding Cas, guide RNA or a DNA encoding the same can be introduced into a host cell by a method similar to the above, according to the host species.

In an embodiment of the present invention, an expression cassette encoding Cas can be inserted, as an exogenous gene, between the two homologous nucleic acid sequences in the exogenous nucleic acid sequence. In such case, since the Cas protein is already expressed in the host cell, as long as a guide RNA specifically recognizing a sequence-specific nuclease-recognizing site is introduced into the host cell, the guide RNA and the Cas form a complex in the host cell, and DSB at the sequence-specific nuclease-recognizing site can occur by the complex. This means that introduction of sequence-specific nuclease in the form of an expression vector into the host cell is not necessary. Therefore, this embodiment is advantageous in that an additional step for removing the expression vector is also unnecessary.

When another sequence-specific nuclease such as ZFN or TALEN or the like is used, an expression cassette encoding the sequence-specific nuclease under the control of an inducible promoter can also be inserted, as an exogenous gene, between the two homologous nucleic acid sequences in the exogenous nucleic acid sequence. In such case, the sequence-specific nuclease is expressed in the host cell by adding an inducer corresponding to the promoter, which can cause DSB at the sequence-specific nuclease-recognizing site. Examples of the inducible promoter include metallothionein promoter (induced by heavy metal ion), heat shock protein promoter (induced by heat shock), Tet-ON/Tet-OFF promoter (induced by addition or removal of tetracycline or a derivative thereof), steroid-responsive promoter (induced by steroid hormone or a derivative thereof) and the like, when a higher eukaryotic cell such as animal cell, insect cell, plant cell or the like is used as a host cell. Expression of the sequence-specific nuclease is induced by adding the corresponding inducer to a medium (or removing the same from a medium) at an appropriate time, and DSB and the subsequent MMEJ or SSA occur by culturing the host cell in the medium in a certain period, thereby a repair of genomic DNA can be achieved. Furthermore, expression of the expression of the sequence-specific nuclease ceases by removal of the expression cassette, thereby the risk of off-target cleavages can be reduced.

### 3. Mutagenesis Using the Method of the Present Invention

As mentioned above, when the host cell used in step (1) of the method of the present invention is provided, one to several nucleotide mutations (e.g., substitution, deletion, insertion, addition) can be introduced into the corresponding endogenous genome sequence in either or both of the homologous nucleic acid sequences.

(i) when the same mutations are introduced into both of the homologous nucleic acid sequences, DSB at the sequence-specific nuclease-recognizing site and the subsequent MMEJ or SSA between the cleaved ends occur by carrying out the method of the present invention, thereby the mutation can be introduced into an endogenous genome sequence corresponding to the homologous nucleic acid sequence in the genome.

(ii) when different mutations (e.g., substitutions with different nucleotides, mutations at different sites and the like) are introduced into both of the homologous nucleic acid sequences, DSB at the sequence-specific nuclease-recognizing site and the subsequent MMEJ or SSA between the cleaved ends occur by carrying out the method of the present invention, thereby two kinds of isogenic cells, in each of which a mutation corresponding to either homologous nucleic acid sequence is introduced into an endogenous genome sequence corresponding to the homologous nucleic acid sequence in the genome, can be obtained.

(iii) when a mutation is introduced into either of the homologous nucleic acid sequences, DSB at the sequence-specific nuclease-recognizing site and the subsequent MMEJ or SSA between the cleaved ends occur by carrying out the method of the present invention, thereby two kinds of isogenic cells, in each of which the mutation is introduced (or not introduced) into an endogenous genome sequence corresponding to the homologous nucleic acid sequence in the genome, can be obtained.

In addition,

(iv) when the host cell used in step (1) of the method of the present invention is provided by homologous recombination, one or more mutations (e.g., substitution, deletion, insertion, addition) can be introduced into an endogenous genome sequence in the aforementioned flanking genome sequence. When the method of the present invention is applied to a host cell in which a mutation is introduced into the flanking genome sequence, DSB at the sequence-specific nuclease-recognizing site and the subsequent MMEJ or SSA between the cleaved ends occur, thereby the mutation can be introduced into the flanking genome sequence in the genome.

For example, by the method of (iii) above, two cell lines that have the same genetic background, with (or without) a mutation in a gene responsible for an inherited disease, can be simultaneously prepared. By using the cell line without the mutation as a control, effects of the mutation on the inherited disease, drug-sensitivity of a cell having the mutation and the like can be more precisely evaluated.

Alternatively, when the method of (i) or (iv) above is applied to a cell having a certain gene mutation (e.g., iPS cell induced from a patient with the mutation or the like), an autogenic cell without the mutation, namely, a cell having a wild-type gene can be prepared. Such autogenic cell reverted to wild-type can be applied as a source of engrafted cells for treating a disease caused by the gene mutation.

### 4. Nucleic Acid for Use in the Method of the Present Invention

The present invention also provides a nucleic acid for use in the method of the present invention (hereinafter also referred to as “the nucleic acid of the present invention”). The nucleic acid is used for preparing the host cell used in step (1) of the method of the present invention.

The nucleic acid of the present invention comprises:

(a) two nucleic acid sequences homologous to a targeted region in a host genome, wherein the 3′ end of one of the nucleic acid sequences and the 5′ end of the other nucleic acid sequence overlap; and

(b) one or more sequence-specific nuclease-recognizing site(s) between the two nucleic acid sequences of (a).

The two nucleic acid sequences of (a) above correspond to a sequence in which the aforementioned homologous nucleic acid sequence is added to the 3′-end of the aforementioned 5′-flanking genome sequence in the method of the present invention, and a sequence in which the homologous nucleic acid sequence is added to the 5′-end of the aforementioned 3′-flanking genome sequence in the method of the present invention. These sequences overlap in the portions of the homologous nucleic acid sequences.

On the other hand, the sequence-specific nuclease-recognizing site(s) of (b) above correspond(s) to one or more sequence-specific nuclease-recognizing site(s) located between the aforementioned two homologous nucleic acid sequences in the method of the present invention.

It is preferable that the two nucleic acid sequences of (a) above contain a sequence-specific nuclease-recognizing site different from the sequence-specific nuclease-recognizing site(s) of (b) above in the 5′- and 3′-flanking genome sequences for the purpose of improvement of homologous recombination efficiency.

It is preferable that the nucleic acid of the present invention contains two or more sequence-specific nuclease-recognizing sites of (b) above, and two of them are substantially adjacent to the two nucleic acid sequences of (a) above, respectively. Herein, the term “substantially” means that the nucleic acid sequence of (a) above is directly ligated with the sequence-specific nuclease-recognizing site, or they are ligated via an intermediate sequence that allows MMEJ or SSA between the overlapping ends of the two nucleic acid sequences of (a) above. In this case, the nucleic acid of the present invention can contain one or more exogenous genes between the two sequence-specific nuclease-recognizing sites substantially adjacent to the nucleic acid sequences of (a) above. Examples of the exogenous gene include those described in the explanation of the method of the present invention.

### 5. Kit for Use in the Method of the Present Invention

The present invention also provides a kit for use in the method of the present invention (hereinafter also referred to as “the kit of the present invention”). The kit comprises:

(a) the nucleic acid of the present invention mentioned above; and

(b) one or two kinds of sequence-specific nuclease(s) specifically recognizing the sequence-specific nuclease-recognizing site (s) contained in the nucleic acid of (a), or nucleic acid(s) that encode the same.

Examples of the sequence-specific nuclease of (b) above include those described in the explanation of the method of the present invention, and are preferably artificial nucleases such as ZFN, TALEN, CRISPR/Cas and the like.

When the nucleic acid of (a) above contains a sequence-specific nuclease-recognizing site different from the sequence-specific nuclease-recognizing site (s) of 4. (b) above in the aforementioned 5′- and 3′-flanking genome sequences, the kit of the present invention can further comprises another sequence-specific nuclease that recognizes and binds to the sequence-specific nuclease-recognizing site for improving homologous recombination efficiency, or a nucleic acid encoding the same.

The present invention is explained in the following by referring to Examples, which are not to be construed as limitative.

### EXAMPLES

### Materials and Methods

### Plasmid Construction

Table 1 provides a list of sequence-verified plasmids used in this study. Full plasmid sequences are available upon request or through Addgene. Primers used for cloning and validation are listed in Table 2.

HPRT1_B NC-TALENs were described previously (Sakuma et al., Genes Cells 18, 315-326, 2013). Avr-TALEN expression vectors with non-repeat-variable di-residue (non-RVD) variations were assembled using the Platinum TALEN method (Sakuma et al., Scientific reports 3, 3379, 2013), into a modified ptCMV-136/63-VR expression vector containing a CAG promoter instead of CMV. The DNA-binding modules were then assembled using the two-step Golden Gate method. Assembled modules were as follows: Left, HD HD NI NG NG HD HD NG NI NG NN NI HD NG NN NG NI NN NI NG; Right, NI NG NI HD NG HD NI HD NI HD NI NI NG NI NN HD NG. TALENs targeting AAVS1 were described previously (Oceguera-Yanez et al., Methods 101, 43-55, 2016).

For CRISPR/Cas9 expression, sgRNA oligos (Table 2) were annealed and cloned into pX330 (Addgene plasmid #42230, a gift from Feng Zhang) linearized with BbsI as previously described (Ran et al., 2013). The resulting plasmids (pX-EGFP-g1, -g2, and -g3) were sequence verified (Table 1).

The HPRT1 SSA reporter vector was used as previously described (Sakuma et al., Genes Cells 18, 315-326, 2013). Additional CRISPR/Cas9 SSA reporter vectors for eGFP sgRNAs were generated by annealing oligos consisting of the protospacer and PAM (Table 2) followed by ligation into pGL4-SSA linearized with BsaI.

To generate the MhAX donor vectors for HPRT1 gene editing, a homology region of 1253 bp surrounding the HPRT1_B TALEN target site was PCR amplified from 201B7 iPSC genomic DNA (Takahashi et al., 2007), cloned into a minimal pBluescript backbone, and sequence verified (p3-HPRT1). The puro-deltaTK selection marker was designed as previously described (Chen and Bradley, 2000), and constructed in an AAVS1 donor vector (Addgene plasmid #22075). InFusion cloning (Clontech) was used to introduce the 2A-puro-deltaTK cassette into the p3-HPRT1 donor vector. Briefly, the p3-HPRT1 vector was inverse-PCR amplified with primers that included all operational sequences for excision and MMEJ repair, including: the eGFP1 protospacer and PAM sequences, appropriately engineered μH, as well as silent and disease-associated mutations (either contained within the μH or within the flanking unique regions as indicated in the text), and terminating with 12-15 nt InFusion overhangs (Table 2). The 2A-puro-deltaTK cassette was amplified such that the T2A and selection marker coding region were in-frame with HPRT exon 3 to give rise to pHPRT1-Ptk-ftsGFP1. To construct the HPRT_{Munich} donor vectors p3-HPRT1-S104R-PdTK-mCh and p3-HPRT1-S104Rf-PdTK-mCh, InFusion primers bearing the modified μH and point mutations were used for PCR (Table 2). Next, the CAG::mCherry reporter was introduced by first using restriction-ligation to clone a CAG::Gateway cassette from pAAVS1-P-CAG-DEST (Addgene plasmid #80490; Oceguera-Yanez et al., Methods 101, 43-55, 2016), followed by Gateway cloning of mCherry.

TABLE 1
Plasmids used in this study.

| | Plasmid | |
|---|---|---|
| Purpose | ID # | Plasmids |
| TALENs | KW228 | PB-CAG-dNC-HPRT1_L-GFP |
| | KW229 | PB-CAG-dNC-HPRT1_R-mCh |
| | TY026 | CAG-Avr-HPRT-LEFT |
| | TY027 | CAG-Avr-HPRT-RIGHT |
| CRISPR/Cas9 | KW532 | pX-EGFR-g1 |
| | KW533 | pX-EGFP-g2 |
| | KW534 | pX-EGFP-g3 |
| | KW817 | pX-APRT-sg1 |
| | KW818 | pX-APRT-sg2 |
| | KW819 | pX-APRT-sg3 |
| | KW820 | pX-APRT-sg4 |
| HPRT Donor Vectors | KW293 | p3-HPRT1 |
| | KW668 | pHPRT1-Ptk-ftsGFP1 |
| | KW836 | p3-HPRT1-S104R-PdTK-mCh |
| | KW838 | p3-HPRT1-S104Rf-PdTK-mCh |
| | KW793 | pHPftsG1-CAG-mCh |
| | KW883 | pHPftsG1-u29-CAG-mCh |
| APRT Donor Vectors | KW827 | pCR4-hAPRT-G |
| | KW1005 | pbG-APRT-J-u32uni-PdTKmCh |
| Donor Cassette | KW999 | pAAVS1-PdTK-CAG-mCh-[uBgIII] |
| Donor Backbone | KW991 | pCAG-eGFP-pA |
| SSA assay (luciferase) | KW850 | pGL4-SSA-eGFP1 |
| | KW859 | pGL4-SSA-eGFP2 |
| | KW862 | pGL4-SSA-eGFP3 |
| MMEJ assay | KW855 | pGL4K-MMEJ-eGFP1-μ0 |
| (luciferase) | KW868 | pGL4K-MMEJ-eGFP1-μ5 |
| | KW856 | pGL4K-MMEJ-eGFP1-μ10 |
| | KW869 | pGL4K-MMEJ-eGFP1-μ15 |
| | KW857 | pGL4K-MMEJ-eGFP1-μ20 |
| | KW870 | pGL4K-MMEJ-eGFP1-μ25 |
| | KW858 | pGL4K-MMEJ-eGFP1-μ30 |
| | KW875 | pGL4K-MMEJ-eGFP1-μ40 |
| | KW876 | pGL4K-MMEJ-eGFP1-μ50 |
| Luciferase Assay | KW208 | pGL4-CMV-luc2 |
| Controls | Promega | pGL4_74_hRlucTK |
| | E6921 | |

TABLE 2
Primers used in Examples.
Donor Construction

| | | | | Size |
|---|---|---|---|---|
| Purpose | Primer ID# | Primer Name | Sequence | (bp) |
| HPRT Homology | dna450 | hHPRT-Fo | GTGCAGTGCAGCAGAATGAT | 1253 |
| Arms | dna411 | hHPRT1Cel-Rev2 | ATTTGTCAAACCTAGCTCCAAAGG | |
| In Fusion (KW668) | dna1649 | HPRT-Ifs | CTCTATGGGTCGACGGGCACGGGCAGCTTGC | 3717 |
| | | | CGGTGGTGACTGTAGATTTTATCAGGTTAAA | |
| | | | GAGCTATTGTGTGAGTAT | |
| | dna1644 | HPRT-Ifas | ACTTCCTCTGCCCTCGGGCACGGGCAGCTT | |
| | | | GCCGG [symbol] TATCTACAGTCATAGGAATGG | |
| | | | ATCTATCACTATTTCT | |
| InFusion Munich | dna1714 | Munich-IF-R | ACTTCCTCTGCCCTCGGGCACGGGCAGCTT | 3713 |
| (KW836, KW838) | | (common) | GCCGG [symbol] TACAATAtCTCTTaAGTCTGAT | |
| | | | AAAATCTACA | |
| | dna1713 | Munich-IF-F | CTCTATGGGTCGACGGGCACGGGCAGCTT | |
| | | (unilateral) | GCCGG [symbol] tAAGAGCTATTGTGTGAGTAT | |
| | | | ATTTAATATATG | |
| | dna1715 | Munich-flank-IF-F | CTCTATGGGTCGACGGGCACGGGCAGCTT | |
| | | (bilateral) | GCCGG [symbol] tAAGAGaTATTGTGTGAGTATA | |
| | | | TTTAATATATG | |
| InFusion of 2A- | dna1642 | 12A-pdtk-Fo | GAGGGCAGAGGAAGTCTTCTAACAT | 1930 |
| puro-delTK | dna1643 | 72A-pdtk-Rev | GTCGACCCATAGAGCCCACCG | |

Operational sequences in MhAX InFusion primers are annotated as follows: underline,InFusion homology; italics,
eGFP1 protospacer; bold italics, PAM; double underline, microhomology; lowercase, mutations.

| | Purpose | Primer ID# | Primer Name | Sequence | Size (bp) |
|---|---|---|---|---|---|
| APRT | Homology Region | dna1692 | hAPRT-HAF | ACTCCTGTCACTTACCCTGA | 1255 |
| | | dna1695 | hAPRT-HAR | CTGGAGGGTTCTAGGTCCTG | |
| | KW1005 InFusion | dna2163 | APRT-Acc65I-A | GCGAATTGGGTACcACTCCTGTCACTTACCCTGACAG | 825 |
| | | | | GCCTAG | |
| | | dna2164 | APRT-J-Acc-B | CTCCGCTGCCAGATCTGGGCACGGGCAGCTTGCCGG | |
| | | | | [symbol] aGCCCAGCAGCTCACAGGCAGCGTTCgTGGTaCC | |
| | | | | TGGGGATGGGAGGGTGA | |
| | | dna2165 | APRT-Acc-C | CCTGCAGCCCAAGCTTGGGCACGGGCAGCTTGCCGG | 570 |
| | | | | [symbol] aGtACCATGAACGCTGCCTGTGAG | |
| | | dna2166 | APRT-Acc65I-D | TCATGGCCGGTACCCTGGAGGGTTCTAGCTCCTGAGG | |
| | | | | TG | |

Operational sequences in MhAX InFusion p imers are annotated as follows: underline, InFusion homology; italics,
eGFP1 protospacer; bold italics, PAM; double underline, mIcrohomology; lowercase, mutations.
PCR Screening

| | | | | Size |
|---|---|---|---|---|
| PCR Reaction | Primer ID# | Primer Name | Sequence | (bp) |
| HPRT1_B mutation | dna309 | hHPRT1Cel-Fo | TITCTGTAGGACTGAACGTCTTGCTC | 305 |
| analysis | dna310 | hHPRT1Cel-Rev | ATCTCACTGTAACCAAGTGAAATGAAAGC | |
| 5′ end (KW668) | dna319 | HPRT1-LaF | GTGGAATTTCTGGGTCAAGGGGAAAGAG | 1134 |
| | dna804 | AAVS1genoS1-2 | GAGCCTAGGGCCGGGATTCTC | |
| 5′ end Munich | dna319 | HPRT1-LaF | GTGGAATTTCTGGGTCAAGGGGAAAGAG | 1158 |
| (KW836, KW838) | dna804 | AAVS1genoS1-2 | GAGCCTAGGGCCGGGATTCTC | |
| Spanning | dna319 | HPRT1-LaF | GTGGAATTTCTGGGTCAAGGGGAAAGAG | 1868 |
| | dna383 | HPRT1-RaR2 | AGGCGAGTTTCTACAAAGATGGACAGG | |
| 3′ end (KW668) | dna930 | TKseq | CCGCGCACCTGGTGCATGAC | 2158 |
| | dna383 | HPRT1-RaR2 | AGGCGAGTTTCTACAAAGATGGACAGG | |
| 3′ end Munich | dna123 | mCherry-F | CCGTAATGCAGAAGAAGACCAT | 1748 |
| (KW836, KW838) | dna383 | HPRT1-RaR2 | AGGCGAGTTTCTACAAAGATGGACAGG | |

PCR Genotyping

| | PCR Reaction | Primer ID# | Primer Name | Sequence | Size (bp) |
|---|---|---|---|---|---|
| APRT | T7E1 | dna1711 | hAPRT-T7F5 | GTCGTGGATGATCTGCTGG | 461 |
| | | dna1712 | hAPRT-T7R5 | TGCCCAAGGCTGATATTTCC | |
| | 5′ end | dna1728 | hAPRT-e1e2-F2 | CTTCCGGCGACGGATGCC | 2287 |
| | | dna804 | T2A-puroJ | GAGCCTAGGGCCGGGATTCTC | |
| | Spanning (non- | dna1796 | SNP-rs3826074-F | TCCTCCATTTCCACCTTCCCTA | 4020 |
| | targeted allele) | dna1865 | hAPRT-HAR2 | GCTTGCTCCCCTAGAAGATG | |
| | 3′ end | dna116 | rBgSp1b | ATGAACAAAGGTGGCTATAAAGAGGTCATC | 876 |
| | | dna1865 | hAPRT-HAR2 | GCTTGCTCCCCTAGAAGATG | |

Southern Blot

| | | | | Size |
|---|---|---|---|---|
| Probe | Primer ID# | Primer Name | Sequence | (bp) |
| mCherry | dna1737 | mCh-probeF | GTTCATGTACGGCTCCAAGG | 505 |
| | dna062 | UniFruitR | TTACTTGTACAGCTCGTCCATGC | |
| HPRT-B | dna1718 | hHPRT-5ext-4F | GCTGAGGATTTGGAAAGGGT | 475 |
| (5′ External) | dna1719 | hHPRT-5ext-4R | GCCAGACATACAATGCAAGC | |

| | Probe | Primer ID# | Primer Name | Sequence | Size (bp) |
|---|---|---|---|---|---|
| APRT | APRT (5′ Internal) | dna1692 | hAPRT-HAF | ACTCCTGTCACTTACCCTGA | 496 |
| | | dna1726 | hAPRT-5int-1R | AGATCATCCACGACGACCAC | |
| Common | mCherry | dna1737 | mCh-probeF | GTTCATGTACGGCTCCAAGG | 505 |
| | | dna062 | UniFruitR | TTACTTGTACAGCTCGTCCATGC | |

sgRNA Cloning

| sgRNA | Primer ILV | Primer Name | Sequence |
|---|---|---|---|
| eGFP-1 | dna1045 | EGFP-gRNA1-Fo | caccgGGGCACGGGCAGCTTGCGGG |
| | dna1046 | EGFP-gRNA1-Rev | aaacCCGGCAAGCTGCCCGTGCCCc |
| eGFP-2 | dna1047 | EGFP-gRNA2-Fo | caccgGATGCCGTTCTTCTGCTTGT |
| | dna1048 | EGFP-gRNA2-Rev | aaacACAAGCAGAAGAACGGCATCc |
| eGFP-3 | dna1049 | E3FP-gRNA3-Fo | caccgGGTGGTGCAGATGAACTTCA |
| | dna1050 | EGFP-gRNA3-Rev | aaacTGAAGTTCATCTGCACCACCc |

lower-case characters indicate overhangs for Bbs I cloning, and the 5′-G

| | sgRNA | Primer ID+190 | Primer Name | Sequence |
|---|---|---|---|---|
| APRT | APRT-sg1 | dna1678 | APRT-Xs1 | caccgCAGGCAGCGTTCATGGTTCC |
| | | dna1679 | APRT-Xas1 | aaacGGAACCATGAACGCTGCCTGc |
| | APRT-sg2 | dna1680 | APRT-Xs2 | caccgGGCAGCGTTCATGGTTCCTG |
| | | dna1681 | APRT-Xas2 | aaacCAGGAACCATGAACGCTGCCc |
| | APRT-sg3 | dna1682 | APRT-Xs3 | caccgAGGCAGCGTTCATGGTTCCT |
| | | dna1683 | APRT-Xas3 | aaacAGGAACCATGAACGCTGCCTc |
| | APRT-sg4 | dna1684 | APRT-Xs4 | caccgCAGCTCACAGGCAGCGTTCA |
| | | dna1685 | APRT-Xas4 | aaacTGAACGCTGCCTGTGAGCTGc |

Lower-case characters indicate overhangs for Bbs I cloning and 5′-G.
SSA Vectors

| protospacer | Primer ID# | Primer Name | Sequence |
|---|---|---|---|
| SSA-eGFP-1 | dna1804 | eGFP1-SSAs | gtcgGGGCACGGGCAGCTTGCCGGTGG |
| | dna1805 | eGFP1-SSAas | cggtCCACCGGCAAGCTGCCCGTGCCC |
| SSA-eGFP-2 | dna1806 | eGFP2-SSAs | gtcgGATGCCGTTCTTCTGCTTGTCGG |
| | dna1807 | eGFP2-SSAas | cggtCCGACAAGCAGAAGAACGGCATC |
| SSA-eGFP-3 | dna1808 | eGFP3-SSAs | gtcgGGTGGTGCAGATGAACTTCAGGG |
| | dna1809 | eGFP3-SSAs | cggtCCCTGAAGTTCATCTGCACCACC |

low er-case characters indicate overhangs for Bsa 1 cloning
Luciferase Assay Vectors

| | protospacer | Primer ID# | Primer Name | Sequence |
|---|---|---|---|---|
| MMEJ Assay | ccdB Cassette (μH | dna142 | CamccdB-F | GGATCCGGTACCGAATTCGCGGCCGCATTAGGCAC |
| | 0-30 bp) | dna1843 | CamccdB-R | GCGGCCGCGAATTCtGTCGACCTGCAGACTGGCTGTG |
| | Common (μH | dna1828 | luc2-eGFP1-uH-F | AGAATTCGCGGCCGCGGGCACGGGCAGCTTGCCGG |
| | 0-30 bp) | | | [symbol] cCGAGGCTAAaGTcGTtGAtTTGGACACCGGTAAG |
| | | | | ACACTGGGT |
| | μ0 | dna1821 | luc2-eGFP1-u0-R | CGGTACCGGATCCGGGCACGGGCAGCTTGCCGG |
| | | | | [symbol] cAAGAAGGGCACCACCTTG |
| | μ5 | dna1822 | luc2-eGFP1-u5-R | CGGTACCGGATCCGGGCACGGGCAGCTTGCCGG |
| | | | | [symbol] cCCTCGAAGAAGGGCACCACCTTG |
| | μ10 | dna1823 | luc2-eGFP1-u10-R | CGGTACCGGATCCGGGCACGGGCAGCTTGCCGG |
| | | | | [symbol] ctTTAGCCTCGAAGAAGGGCACCACCTTG |
| | μ20 | dna1825 | luc2-eGFP1-u20-R | CGGTACCGGATCCGGGCACGGGCAGCTTGCCGG |
| | | | | [symbol] cAaTCaACgACtTTAGCCTCGAAGAAGGGCACCACCT |
| | | | | TG |
| | μ30 | dna1827 | luc2-eGFP1-u30-R | CGGTACCGGATCCGGGCACGGGCAGCTTGCCGG |
| | | | | [symbol] cCCGGTGTCCAAaTCaACgACtTTAGCCTCGAAGAAG |
| | | | | GGCACCACCTTG |
| | pGLK-CMV-luc2 | dna1848 | luc2-uH-F2 | CGAGGCTAAaGTcGTtGAtTTGGACACCGGTAAGACACT |
| | (μH 40,50 bp) | | | GGGTGTGAACCAGCGCGGCGAGCTGTGCGT |
| | | dna1847 | luc2-u40plus-R2 | cAGTGTCTTACCGGTGTCCAAaTCaACgACtTTAGCCTC |
| | | | | GAAGAAGGGCACCACCTTGCCTACTGCGCCA |
| | Common (μH | dna1844 | eGFP1-Camccd13- | ACgACtTTAGCCTCGg [symbol] CCGGCAAGCTGCCCGTGC |
| | 40, 50 bp) | | R2 | CC GCGGCCGCGAATTCTGTCGACCTGCAGACTGGCT |
| | | | | GTG |
| | μ40 | dna1845 | eGFP1-CamccdB- | ACCGGTAAGACACTg [symbol] CCGGCAAGCTGCCCGTGC |
| | | | u40-F | CC GGATCCGGTACCGAATTCGCGGCCGCATTAGGCA |
| | | | | C |
| | μ50 | dna1846 | eGFP1-CamccdB- | ACCGGTAAGACACTgGGTGTGAACCg [symbol] CCGGCAA |
| | | | u50-F | GCTGCCC GTGCCCGGATCCGGTACCGAATTCGCGGC |
| | | | | CGCATTAGGCAC |

Lower-case characters indicate overhangs for Bsa I cloning in SSA primers, and silent mutations in MMEJ primers. Operational
sequences In MMEJ Assay primers are annotated as follows: underline, InFusion homology; italics, eGFP1 protospacer; bold italics,
PAM; double underline, microhomology. For p40 and p50 assembly, InFusion sites were within the engineered microhomology.
Sequencing

| Application | Primer ID# | Primer Name | Sequence | | |
|---|---|---|---|---|---|
| Targeted 5′ arm | dna319 | HFRT1-LaF | GTGGAATTTCTGGGTCAA GGGGAAA GAG | | |
| junctions | dna1733 | HPRT-seq2 | CCTTTGCCCTCATGTTTCAT | | |
| Targeted 5′ arm | dna116 | rBgSp1b | ATGAACAAAGGTGGCTATAAAGAGGTCATC | | |
| junctions | dna117 | rBgSp2c | CCCAGTCATAGCTGTCCCTCTTCTCTTATG | | |
| SSA vectors | dna197 | SSAseq-Fo | CTCAGCAAGGAGGTAGGTGAGG | | |
| | dna198 | SSAseq-Rev | TGATCGGTAGCTTCTTTTGCAC | | |
| cloned sgRNA | dna790 | U6-fwd | GAGGGCCTATTTCCCATGATTCC | | |
| Exon | | | Fwd Primer | | Amplicon |
| no. | ENSEMBL exon ID | Length | Name | Sequence | Size |
| 1 | ENSE00001913528 | 186 | dna1871 | CAGGGAGCCCTCTGAATAGGA | 536 |
| | | | dna1872 | GTGACGTAAAGCCGAACCC | |
| 2 | ENSE00003489858 | 107 | dna1873 | TAGTAGAGACGGGATITCACC | 466 |
| | | | dna1874 | AGAACAGCTGCTGATGTTTGA | |
| 3 | ENSE00003623041 | 184 | dna1875 | TTGGTGTGGAAGTTTAATGACTAAG | 385 |
| | | | dna1876 | ATCTCACTGTAACCAAGTGAAATG | |
| 4 | ENSE00003674574 | 66 | dna1877 | TCTAGTCATTCATTTCAGGAAACCT | 339 |
| | | | dna1878 | ATTGATTGAAAGCACACTGTTACT | |
| 5 | ENSE00003522510 | 18 | dna1879 | AGCAGATGGGCCACTTGTTTA | 252 |
| | | | dna1880 | TGGCTTACCTTTAGGATGGTG | |
| 6 | ENSE00003576599 | 83 | dna1881 | GGGCCAGATGATATAGATTCCA | 332 |
| | | | dna1882 | TGACAGTTGAAAACATTTATCCTTA | |
| 7, 8 | ENSE00003676328, | 47, 77 | dna1883 | TGCTGCCCCTTCCTAGTAATC | 651 |
| | ENSE00003495603 | | dna1884 | GCCAGGTTCCAGTTCTAAGGA | |
| 9 | ENSE00001904310 | 639 | dna1885 | TGTGATAGACTACTGCTTTGTTTTC | 1019 |
| | | | dna1886 | CCGCCAACCCATTCTACC | |

KAPA Taq Extra

| | Exon | | | Fwd Primer | | Amplicon |
|---|---|---|---|---|---|---|
| Gene | no. | ENSEMBL exon ID | Length | Name | Sequence | Size |
| APRT | 1 + 2 | ENSE00002586104, | 125, 107 | dna1728 | CTTCCGGCGACGGATGCC | 640 |
| | | ENSE00001503918 | | dna1729 | CTCAATCTCACAACCCTTCCCG | |
| | 3, 4, 5 | EN5E00001503917, | 134, 79 | dna1740 | CATGGGGAGAGGAAGGTGT | 1255 |
| | | ENSE00003473485, | 143 | dna1741 | GTACAGGTGCCAGCTTCTCC | |
| | | ENSE00002584924 | | | | |

### SSA Assay

SSA assays were carried out as previously described (Ochiai et al., 2010). Briefly, DNA mixtures containing 200 ng each of TALEN or CRISPR/Cas9 nuclease expression vectors, 100 ng of the appropriate pGL4-SSA target vector, and 20 ng pGL4_74_hRlucTK Renilla reference vector were prepared in 25 μL of Opti-MEM I reduced-serum medium (Invitrogen) in a 96 well plate. 25 μL of Opti-MEM I containing 0.7 μL of Lipofectamine 2000 (Invitrogen) was then added, and incubated at room temperature for 30 min. HEK293T cells (Thermo Scientific) were then added at a density of 4×10⁴ cells per 100 μL in DMEM containing 15% FBS, and cultured at 37° C., 5% CO₂ for 24 hr. To assay luciferase activity, plates were first equilibrated to room temperature before replacing 75 μL of growth medium with 75 μL of Dual-Glo reagent (Promega). After 10 min incubation, 150 μL of reaction was transferred to a white microtitre plate, and luminescence (1 sec) was read on a Centro LB960 (Berthold) or 2104 EnVision Multilabel Plate Reader (Perkin Elmer). Following the addition of 50 μL Stop reagent and 10 min incubation, Renilla luminescence was similarly read. Activity was calculated by the ratio of Firefly/Renilla intensity.

ESC and iPSC Culture

Undifferentiated human ESCs and iPSCs were maintained under feeder-free conditions as described previously (Kim et al. 2016). Briefly, H1 hESCs (Thomson et. al., 1998) and 1383D6 iPSCs were cultured on recombinant human Lamin-511 E8 fragment (iMatrix-511, Nippi) coated 6-well tissue culture plates (0.5 microgram/cm²) in StemFit AK03 or AK02N (AJINOMOTO) medium. For passaging, cells were detached by treatment with 300 microlitters Accumax (Innovative Cell Technologies, Inc.) at 37° C. for 10 min, followed by gentle mechanical dissociation with a pipette. To collect the cells, 700 microlitters of culture medium containing 10 micromolars ROCK inhibitor, Y-27632 (Wako) was added. Cells were counted using trypan blue exclusion on a TC20 (Bio-Rad). Typically, 1-3×10³ cells per cm² were seeded on each passage in media containing Y-27632. After 48 hr culture, the medium was changed without Y-27632.

Five to seven days after plating, the cells reached 80-90% confluency and were again prepared for passage. For making frozen hiPSC stocks, cells were resuspended at a density of 1×10⁶ viable cells per 1 mL STEM-CELLBANKER (Takara) and 200-500 microlitters of cell suspension (2-5×10⁵ hiPSC) was transferred to a cryogenic tube. Stock vials were defrosted onto iMatrix-511 coated 6-well tissue culture plates (one vial per 10 cm²) in StemFit AK03 or AK02N medium containing Y-27632.

Maintenance of 409B2 (Okita, et. al., 2010) was carried out on SNL feeder cells (Tsubooka, et. al., 2011) in Primate ES Cell medium (ReproCELL). For passaging, SNL feeder cells were detached from the well by incubation with 300 microlitters CTK solution containing 1 mg/ml collagenase, 0.25% trypsin, 20% KSR, and 1 mM CaCl₂ in Dulbecco's phosphate buffered saline (DPBS) Mg²⁺ and Ca²⁺ free (Nacalai Tesque) for 2 min at room temperature. CTK solution was then removed and wells were washed twice with 2 mL DPBS. 1 mL of Primate ES Cell medium (ReproCELL) supplemented with Recombinant Human FGF-basic (PEPROTECH) was added and colonies were collected with a cell scraper and dissociated into small clumps by pipetting up and down a few times throughout the entire well. The split ratio was ˜1:5 to a fresh SNL feeder-coated plate.

HPRT Knockout with TALENs

HPRT1 knockout experiments using NC-TALENs in 40952 iPSCs were carried out on SNL feeders with delivery of DNA by Neon (Invitrogen) electroporation as previously described (Sakuma et al., Genes Cells 18, 315-326, 2013). TALEN evaluation assays and HPRT1 knockout experiments using Avr-TALEN in H1 ESCs and 1383D6 iPSCs were carried out under feeder-free conditions with delivery of DNA by NEPA21 (Nepa Gene Co., Ltd) as previously described (Oceguera-Yanez et al., Methods 101, 43-55, 2016). Briefly, CAG-dNC-HPRT1 TALENs (3 μg each) or CAG-Avr-HPRT TALENs (3 μg each) were transfected by NEPA21 electroporation into 1×10⁶ cells in a single-cell suspension. Electroporated cells were plated at a density of 1-5×10⁵ cells/60 mm culture dish. Two days after electroporation, 6-thioguanine (6-TG, 20 μM; Sigma-Aldrich) selection was initiated, with daily feeding over a period of 7-10 days. For population analyses, at cultures of at least 50-300 colonies were pooled and passaged once before genomic DNA preparation. For clonal analyses, iPSC colonies were isolated manually with a micropipette and cultured, processed and stored frozen in 96-well format as previously described (Kim et al., 2016). Selected clones were defrosted and expanded for permanent storage in liquid nitrogen.

iPSC Gene Targeting

Gene targeting was carried out essentially as described (Oceguera-Yanez et al., Methods 101, 43-55, 2016). Briefly, nuclease expression vectors (1 μg for CRISPR, 1 μg each for TALENs) and donor vectors (3 μg) were transfected by NEPA21 electroporation into 1×10⁶ cells in single-cell suspension. Electroporated iPSCs were plated at a density of 1-5×10⁵ cells per 60 mm culture dish in Stemfit media containing Y-27632. Two days after electroporation, Y-27632 was removed and 0.5 μg/mL puromycin (Sigma-Aldrich) added, with daily feeding over a period of 7-10 days. Clones were isolated manually with a micropipette and processed in 96-well format as described above.

### Cassette Excision

To initiate cassette excision, 1 μg of pX-EGFP-g1 expression vector was transfected by NEPA21 electroporation into 1×10⁶ cells in single-cell suspension, and plated at a density of 1-5×10⁵ cells per 60 mm culture dish in Stemfit media containing Y-27632. Two days after electroporation, Y-27632 was removed.

Cassette excision enriched by HAT selection (1×) was carried out with daily feeding over a period of 7-10 days. Clones were isolated manually and processed in 96-well format as described above.

For cassettes including a fluorescence reporter, enrichment of cassette-excised mCherry negative cells by FACS was performed. iPSCs electroporated with pX-EGFP-g1 were plated as usual and allowed to recover in the absence of selective pressure. After 6 days, cells were subjected to FACS sorting as described below. Recovered mCherry-negative cell populations were counted and plated at clonal density in the presence or absence of HAT (1×). Clones were isolated manually and processed in 96-well format as described above.

### Flow Cytometry and Cell Sorting

For routine measurement of GFP or mCherry fluorescence intensities, 3.0×10⁵ cells were suspended in FACS Buffer (DPBS supplemented with 2% BSA) and analyzed using a BD LSRFortessa Cell Analyzer (BD Biosciences) with BD FACSDiva software (BD Biosciences). mCherry fluorescence intensities of clones targeted with p3-HPRT1-S104R-PdTK-mCh (unilateral S104R Munich mutation) or p3-HPRT1-S104Rf-PdTK-mCh (bilateral S104R Munich mutation) were measured in 96-well format on a MACSQuant VYB (Miltenyi Biotec).

For the isolation of cassette-excised mCherry-negative iPSCs, cells were harvested as a single-cell suspension in FACS Buffer at a density of ˜1×10⁶ cells per mL and filtered through a cell-strainer to remove clumps. After setting gates for singlets, the mCherry-negative cell population was collected on a BD FACSAria II cell sorter (BD Biosciences) into Stemfit AK02N medium containing 20 μM Y-27632. Sorting efficiencies were determined using a BD LSRFortessa Cell Analyzer.

Flow cytometry data were analyzed and generated by FlowJo software (Tree Star).

### Crystal Violet Staining

Plates of iPSCs from confluent or drug-selected cultures were washed twice with ice-cold DPBS and fixed by ice-cold methanol (Nacalai Tesque) for 10 min at room temperature. The methanol was removed and sufficient crystal violet solution (HT90132, Sigma-Aldrich) was added to cover the bottom of the plate. After 10 min incubation at room temperature, the staining solution was removed and the plates were gently rinsed with ddH₂O. After complete drying at room temperature, whole well images were acquired with a STYLUS XZ-2 (OLYMPUS) camera.

### Genomic DNA Isolation

Genomic DNA for PCR screening and sequencing was extracted from 0.5-1×10⁶ cells using a DNeasy Blood & Tissue Kit (Qiagen) according to the manufacturer's instructions. Genomic DNA for Southern blotting was extracted from one confluent well of a 6-well dish (˜1-3×10⁶ cells) using lysis buffer (100 mM Tris-HCl, pH 8.5, 5 mM EDTA, 0.2% SDS, 200 mM NaCl, and 1 mg/mL Proteinase K), followed by standard phenol/chloroform extraction, ethanol precipitation, and resuspension in TE pH 8.0. For high-throughput Southern blotting or PCR screening, genomic DNA was extracted in 96-well format (Ramirez-Solis et al., 1992) using plate lysis buffer (10 mM Tris-HCl, pH 7.5, 10 mM EDTA, 0.5% sarcosyl, 10 mM NaCl, and 1 mg/mL Proteinase K) followed by direct ethanol precipitation and re-suspension in restriction digestion mix or TE pH 8.0.

### PCR Genotyping

Primer design for exons 1-9 of HPRT1 (Accession NG_012329.1) was performed using the NCBI Primer-BLAST with optional settings for human repeat filter, SNP handling, and primer pair specificity checking to H.sapiens (taxid:9606) reference genome (Table 2). For H1 ESCs and 1383D6 iPSCs exons 1-9 were amplified from genomic DNA with KAPA Taq Extra using the following protocol (98° C. for 10 sec, 59° C. for 15 sec, 68° C. for 4 min)×30 cycles, 4° C. hold, and sequenced.

For gene targeting, puro-resistant clones were screened by PCR to verify the 5′ and 3′ targeting junctions. Primers outside of the donor vector homology arms and transgene specific primers were used as described in FIGS. 9 and 12, and Table 2. PCR was carried out with KAPA Taq Extra using the following protocol (98° C. for 10 sec, 59° C. for 15 sec, 68° C. for 4 min)×30 cycles, 4° C. hold. Sequencing of the junction regions was used to ensure the fidelity of the flanking μH and CRISPR protospacers.

HPRT1_B TALEN-induced mutations spectra and MMEJ repair rates following excision of the targeting cassette were screened from pooled or clonal genomic DNA preparations using AmpliTaq 360 (ABI) 95° C. for 10 min (95° C. for 30 sec, 57° C. for 30 sec, 72° C. 60 sec)×30 cycles, 72° C. 7 min 4° C. hold, with primer set dna309/310. PCR products from clones were sequenced directly using the same primers, while PCR products from pools were cloned using a TOPO TA Cloning Kit (Invitrogen), and then individually sequenced from the resulting bacterial colonies following PCR amplification with T3/T7 primers.

In order to verify deposition of the Silent mutation following excision with unilaterally or bilaterally mutant μH, genomic DNA was amplified using primers dna1720/411. Cleaved amplicons were resolved by gel electrophoresis following treatment with or without AflII restriction enzyme.

### Sequencing

PCR products were treated with ExoSAP-IT (Affymetrix) prior to sequencing. DNA sequencing was performed using BigDye Terminator v3.1 Cycle Sequencing Kit (Applied Biosystems), purification by ethanol precipitation, and run on a 3130×1 Genetic Analyzer (Applied Biosystems). Sequence alignments were performed using Sequencher v5.1 (Genecodes) or Snapgene v3.1.4 or greater (GSL Biotech LLC.). Sequence trace files with poor base calling confidence were excluded from further analyses.

### TIDE Analysis

Populations of iPSCs consisting of approximately 50 clones (H1) or 200 clones (1383D6) were pooled and harvested for genomic DNA and amplified as described above. TIDE analysis of mixed sequences was performed using the online tool at https://tide.nki.nl/ (Brinkman et al., 2014). Sequence data from 1383D6 iPSCs or H1 ESCs was used as a reference. Since TIDE is designed for CRISPR/Cas9, and TALENs induce DSBs at an undetermined position within the spacer, we positioned the predicted breakpoint at the 5′ end of the spacer, adjacent the HPRT1_B TALEN-L binding site (ATTCCTATGACTGTAGAT̂TTT), where base-calling confidence initially dropped co-incident with visibly mixed sequence. The deletion size window was extended to 20 bp to accommodate larger deletions. The remaining parameters were set to default or allowed to adjust automatically based on the properties of the sequence trace files provided.

### Southern Blotting

The HPRT-B and mCherry probe fragments were prepared from a genomic or plasmid PCR amplicon, respectively (Table 2), while the TK probe was prepared from a plasmid restriction fragment. DIG labeled dUTP (Roche) was incorporated by PCR amplification using ExTaq (Takara) in the case of HPRT-B and mCherry or random priming in the case of TK, according to the manufacturer's instructions.

Genomic DNA (5-10 μg) was digested with 3- to 5-fold excess restriction endonuclease overnight in the presence of BSA (100 μg/mL), RNaseA (100 μg/mL) and spermidine (1 mM). Digested DNA fragments were separated on a 0.8% agarose gel, depurinated, denatured, and transferred to a Hybond N+ nylon membrane (GE Healthcare) using 20×SSC. The membrane was UV crosslinked, pre-hybridized, and incubated with 150 ng/mL digoxigenin (DIG)-labeled DNA probe in 4 mL DIG Easy Hyb buffer (Roche) at 42° C. overnight with constant rotation. After repeated washing at 65° C. (0.5×SSC; 0.1% SDS), the membrane was blocked (DIG Wash and Block Buffer Set, Roche) and alkaline phosphatase-conjugated anti-DIG antibody (1:10,000, Roche) was applied to a membrane. Signals were raised by CDP-star (Roche) and detected by ImageQuant LAS 4000 imaging system (GE Healthcare).

### Microscopy

Phase-contrast and fluorescence images were acquired on a BZ-X710 (KEYENCE) using appropriate filters and exposure times.

### Cell Growth Measurement

iPSC lines were plated 3×10⁴ cells per 6 well culture dish, and grown for 2 days without HAT, followed by 2 additional days with or without HAT. Cells were harvested on days 2, 3 and 4 post-plating, and re-suspended in 100 μL of AK02. An 11 microlitters aliquot of cell suspension was mixed 1:1 with Trypan Blue Stain 0.4% (Gibco) by gentle pipetting, and 10 microlitters were applied to each side of a Counting Slide (Bio-Rad). Cell numbers were determined with the TC20 Automated Cell Counter (Bio-Rad).

### Western Blotting

For HPRT protein analysis, total cell lysates were prepared by boiling 1×10⁶ cells for 10 min in 100 μL NuPAGE LDS Sample Buffer (1×) (Thermo Fisher Scientific) containing DTT at a final concentration of 50 mM. Lysates were resolved on Bis-Tris gels, and probed using HPRT (F-1, sc-376938, 1:200, Santa Cruz) and Anti-actin (A2066, 1:5,000, Sigma Aldrich) antibodies. Goat anti-rabbit IgG-HRP (Santa Cruz: sc-2004) and Anti-Mouse IgG, HRP-Linked Whole Ab Sheep (GE Life Science:NA931-100UL) secondary antibodies for HPRT and Anti-actin, respectively, were used at 1:5000 dilution. Signals were raised using ECL Prime Western Blotting Detection Reagent (GE Healthcare) and detected on an ImageQuant LAS 4000 imaging system (GE Healthcare).

### Metabolome Analysis

Medium samples were analyzed using capillary electrophoresis time-of-flight mass spectrometry (CE-MS) as described (Wakayama, et. al., 2015). For sample preparation, 1.5×10⁵ cells from the indicated iPSC clones were seeded in 150 μL of AK02 medium containing ROCKi (10 μM) per well of a 96 well plate and cultured at 37° C., 5% CO₂. The next day, the medium was replaced with 150 μL of fresh AK02 medium without ROCKi. Media-only reference samples were prepared and similarly incubated at 37° C., 5% CO₂. After 24 hr, 100 μL of spent medium was collected and mixed with 400 microlitters of methanol containing L-methionine sulfone (Wako), MES (Dojindo), and CSA (Wako) internal standards (200 micromolars each). Following the addition of 200 microlitters Milli-Q ultrapure water, the samples were extracted with 500 microlitters chloroform. The aqueous layer was subjected to 5 kDa ultrafiltration (HMT) and lyophilized (LABCONCO). Lyophylized samples were resuspended in 50 microlitters Milli-Q ultrapure water containing 3-Aminopyrrolidine (Sigma Aldrich) and Trimesate (Wako) internal standards (200 micromolars each) before analysis. The data were analyzed and quantified using in-house software (Master Hands-2.17.1.11) developed particularly for CE-MS-based metabolomic data analysis.

### Results

### MMEJ Biases DSBR Outcomes Following TALEN Cleavage of the HPRT1 Locus

Gene disruption using programmed endonucleases relies on cellular error-prone repair pathways such as nonhomologous end joining (NHEJ) to produce random insertion and deletion (indel) mutations. We previously exploited this phenomenon to disrupt HPRT enzyme function in 201B7 human female iPSCs in order to assess the activities of modified TALEN architectures (Sakuma et al., Genes Cells 18, 315-326, 2013). In that assay, transient transfection of TALENs modeled after HPRT1_B (Cermak et al., 2011) which target exon 3 of the human HPRT1 gene (FIG. 1A), followed by enrichment for 6-thioguanine resistance (6-TG^{R}), revealed a recurring mutation comprised of 17 deleted bases (delta17). TALEN-mediated disruption of HPRT1 in another female iPSC line (409B2) reproduced the delta17 allele at a frequency of ˜25% (FIG. 2). NHEJ outcomes may be biased by short direct sequence repeats in an alternative repair pathway deemed microhomology-mediated end joining (McVey and Lee, Trends in genetics: TIG 24, 529-538, 2008). We therefore used a custom Python script based on (Bae et al., 2014) to detect microhomology (μH) at the expected DSB site. The script predicted a 5 bp μH (μ5: ‘GACTG’) lying within the left TALEN (TALEN-L) binding site and the intervening spacer region, separated by 12 bp of nonhomologous sequence (FIG. 1A). Further examination revealed a second μH of 3 bp (μ3: ‘AGA’) adjacent to μ5, separated by only one variant base (T or A), resulting in an imperfect compound μH of the structure ‘GACTGWAGA’, where W=T/A (hereafter referred to as μ5W3). These observations suggested a biased repair pathway through MMEJ which warranted further investigation.

Prior to assessing MMEJ at the target site, we made three marked technical improvements in our HPRT1 TALEN assay. First, considering the HPRT1 locus is X-linked, we chose to employ male 1383D6 iPSCs (Oceguera-Yanez et al., Methods 101, 43-55, 2016) and H1 ESCs (Thomson et al., 1998), neither of which bear deviations from the reference human genome in HPRT1 exons 1-9 (data not shown). Although female iPSC lines grown under conditions that promote bi-allelic X-activation (X^{a}/X^{a}, Tomoda et al., Cell stem cell 11, 91-99, 2012) demonstrated the robustness of nuclease cleavage (Sakuma et al., Genes Cells 18, 315-326, 2013), a single HPRT1 copy in male lines would help clarify the NHEJ mutation spectra. Second, we adapted our assay to feeder-free conditions (Nakagawa et al., 2014), which improved clonal analyses by permitting single cell passage, cloning, and expansion in 96-well format (Kim et al., 2016). Moreover, eliminating HPRT1-negative SNL feeders (Okita et al., 2011) significantly improved the kinetics of drug toxicity for both 6-TG and HAT selection by avoiding cross-feeding or feeder sensitivity, respectively. Third, whilst maintaining the same target sequences (Cermak et al., 2011), HPRT1_B TALENs were updated from a truncated Xanthomonas oryzae pv. (PthXo1)-based TALE scaffold (Sakuma et al., Genes Cells 18, 315-326, 2013a) to X. campestris pv. vesicatoria (AvrBs3)-based +136/+63 TALE architecture (Christian et al. 2010; Sakuma et al., Scientific reports 3, 3379, 2013) and expressed from a new CAG promoter-driven expression vector (Table 1). These combined vector modifications resulted in a 3-fold increase in cleavage activity for AvrHPRT1_B TALENs as measured by single-strand-annealing assay (Sakuma et al., Scientific reports 3, 3379, 2013; FIG. 3A). Enhanced genome cleavage activity was also demonstrated by improved 6-TG^{R} colony formation following transfection of 1383D6 male iPSCs (FIG. 3B).

With these improvements, we set out to explore the spectrum of mutations induced by AvrHPRT1_B TALENs in male iPSCs. We estimated allele frequencies in a bulk population of 6-TG^{R} male iPSCs by employing computational sequence trace decomposition from mixed PCR amplicons (TIDE, Brinkman et al., 2014). In the sequence trace file, overlapping peaks were observed immediately following μ5W3, with a preceding T/A overlay at position ‘W’ (FIG. 4A-C). Amongst a variety of minor deletion alleles, delta17 was found to be significantly overrepresented (63.5%, FIG. 4D), strongly supporting MMEJ through μ5W3. The TIDE result was verified at a similar frequency in male H1 human ES cells (43.9%, FIG. 4E-G). In order to exclude the possibility that this apparently high rate of MMEJ repair in the population was an artifact of PCR bias, we isolated 6-TG^{R} iPSC clones and performed Sanger sequencing of exon 3 (FIGS. 1B and 5). Clonal analysis revealed deletions as the most common NHEJ outcome (83%), amongst which the delta17 allele comprised the majority (69%), consistent with the population-based TIDE analysis. The delta17 alleles could be further subdivided according to the imperfection in μ5W3 at a ratio of 5(T):15 (A) (FIG. 1C), presumably dictated by more frequent use of the upstreamμ5 for repair, and a concordant loss of the intervening ‘TAGA’ sequence. Both Δ17 deletion types produce a −1 frame shift which results in three (D98E, F99L, I100L for HPRT^{Δ17T}) or four (V97E, D98E, F99L, I100L for HPRT^{Δ17A}) missense mutations terminating in a nonsense mutation (fsTer101), resulting in loss of HPRT function as measured by resistance to 6-TG and sensitivity to HAT (FIG. 6A), with no additional effects on clone morphology or growth rate under normal culture conditions. Analysis of the TALEN-mediated HPRT1 knockout data led us to two key conclusions (FIG. 1D): first, that common MMEJ events reproducibly result in high-fidelity deletion of intervening sequence, and second, that MMEJ between imperfect μH (μ5W3) leads to alternate yet predictable allelic outcomes.

### Point Mutation Deposition Using a Cassette Designed for MMEJ-Assisted Excision

Inspired by TALEN-mediated HPRT1 disruption (FIG. 1), we reasoned that by engineering endogenous sequences as duplicated μH such that they flank an antibiotic selection marker, we could recruit the cell to employ MMEJ to repair nested DSBs, resulting in scarless excision and locus restoration (FIG. 7A). To demonstrate this microhomology-assisted excision (MhAX) technique, we chose to target HPRT1 exon 3 using a puro-ΔTK antibiotic counter-selection cassette (a fusion of puromycin to truncated thymidine kinase) with the intent to track both gene targeting and excision steps. Since HPRT1 is expressed in human iPSCs, we employed the cassette as a 2A-peptide linked promoterless gene-trap; an approach similar to that used for background-free AAVS1 targeting (Oceguera-Yanez et al., Methods 101, 43-55, 2016), but lacking a splice-acceptor sequence in favor of in-frame insertion into the HPRT1 open reading frame (FIG. 8A).

In order to generate DSBs flanking the marker, we chose to employ CRISPR/Cas9 rather than TALEN, exploiting multiple advantages including: a unified Cas9 protein and sgRNA plasmid expression system (Ran et al., 2013) and defined endonuclease breakpoints (Jinek et al., 2012). We considered candidate sgRNAs with proven activity which were predicted to have few off-target sites in the human genome, and chose to initially focus on three sgRNAs targeting the GFP gene of A. victoria, already shown to have high activity and low toxicity in human U2OS osteosarcoma cells (Fu et al., 2014). A plasmid-based SSA assay measuring luciferase repair in HEK293T cells (Ochiai et al., 2010) determined relative activities for each sgRNA (FIGS. 9A and B), with eGFP sgRNA1 found to be the most potent, verifying the results of the original report (Fu et al., 2014). We further determined the activity of the eGFP sgRNA series using a genome cleavage assay in human iPSC (FIG. 9C), which measures disruption of a constitutive CAG::GFP transgene targeted to the AAVS1 locus (Oceguera-Yanez et al., Methods 101, 43-55, 2016). FACS analysis for GFP 5 days after transfection with the nuclease without enrichment showed a 7.4% GFP negative fraction for sgRNA1, proving its utility in cleaving the genome of human iPSCs. No overt cytotoxicity was observed for any of the sgRNAs in either assay. Based on these data, we positioned the eGFP-1 protospacer flanking the cassette in a divergent orientation, such that the PAMs and upstream cleavage sites were proximal to the engineered μH (FIGS. 7A and 8A).

In designing the flanking μH, we made use of the native μ5T3 sequence (FIG. 1A). We engineered silent mutations in the right homology arm of the donor vector to demonstrate scarless deposition and while also obstructing possible interactions between μ5T3 and μ5A3 (FIGS. 7A and 8A). High-throughput screening and computational analysis of sgRNA libraries (Doench et al., 2014; Doench et al., 2016) has revealed that a ‘G’ nucleotide positioned downstream of the PAM is unfavorable for Cas9 activity. We therefore intentionally lengthened the μH such that each nested eGFP-1 PAM would be flanked by a ‘T’ or an ‘A’ nucleotide. Finally, for 2A-puro-ΔTK expression, μ5T3 was adjusted to maintain the open reading frame, which now included the 5′ flanking eGFP1 protospacer. Thus, the final flanking μH was a contiguous 11 bp sequence, ‘TGACTGTAGAT’. This μH was engineered into the 3′ end of the left and 5′ end of the right homology arms of an HPRT1 donor vector by PCR amplification, such that they flanked the selection marker and CRISPR target sites in tandem (FIG. 7A).

Gene targeting of the prototype MhAX selection marker into 1383D6 male iPSCs was stimulated using HPRT1_B TALENs followed by selection for targeted clones with puromycin. All clones were pre-screened by PCR followed by Sanger sequencing of targeting junctions (FIG. 8B), and subsequently genotyped by Southern blot using internal TK and external HPRT probes to rule out random integration and prove HPRT knock-in, respectively (FIG. 8C). Positive colonies were drug-selected to functionally verify HPRT1 knockout (6-TG^{R} and HAT^{S}; FIG. 7B, middle) and ensure purity without parental iPSC contamination at <1 in 10⁶ cells by colony formation in HAT medium.

In order to excise the selection marker, clone 016-A3 was transfected with an expression vector for Cas9 and eGFP1 sgRNA (pX-EGFP-g1) followed by HAT selection for colony formation. Colony formation was specific to, and dependent on, treatment with the eGFP1 sgRNA, as eGFP2 sgRNA did not induce HAT^{R} colony formation (FIG. 8D), nor did spontaneous reversion of the allele occur even after multiple passages (data not shown). Selection against the cassette using FIAU was ineffective, perhaps because of low endogenous HPRT1 expression driving 2A-puro-ΔTK, analogous to our experience with low-level neo expression from gene-trapping the AAVS1 locus (Oceguera-Yanez et al., Methods 101, 43-55, 2016). In any case, the resulting HATR clones were also sensitive to puro and 6-TG, suggestive of scarless excision (FIG. 7B). Southern blot analysis indicated reconstitution of the HPRT1 locus, while probing for the selection marker (TK probe) revealed no banding in excised clones, proving that the cassette was removed without re-integration (FIG. 7C).

Genomic PCR and sequencing (FIGS. 7D and E) revealed that greater than 93% (42/45) of all clonally isolated HAT^{R} iPSCs were repaired as predicted to occur through MMEJ of the engineered μH. All 42 clones bore the engineered silent mutations, indicating that they were distinct from parental 1383D6 iPSCs and arose as a result of MMEJ. As NHEJ of the flanking DSBs resulting in indels is expected, we explored repair fidelity in the absence of HAT selective pressure. Clone 016-A3 was transfected with pX-eGFP-g1 and total genomic DNA was collected from HAT-unselected populations followed by target region amplification by PCR and sequencing of TA-cloned products. In the unselected population, multiple clones presented fusion of the two eGFP1 protospacer breakpoints with or without various additional short indels (FIG. 7E, right, and data not shown), inferring classic NHEJ as the repair pathway. Importantly, ˜10.5% of sequences (9/86) bore the correct deletion size for MMEJ excision, and represented a perfectly reconstituted HPRT coding sequence predicted for MMEJ-mediated repair (FIG. 7E, left). Thus, we established MhAX as a high-fidelity scarless selection marker excision method and novel approach to deposit designer point mutations in the genome.

### Unilateral μH Mutations Allow for the Coincident Isolation of Isogenic Controls

Considering our observations for imperfect μ5W3 repair at the HPRT1 locus (FIG. 1), we surmised that the duality of outcomes could be intentionally exploited to produce both mutant and control iPSC clones from a single experiment. We therefore chose to focus on re-creating the HPRT_{Munich} partial enzyme deficiency (Wilson et al., J Biol Chem 256, 10306-10312, 1981) caused by a C-to-A transversion mutation (312C>A; rs137852485) (Cariello et al., 1988), located within exon 3 of HPRT1 neighboring the AvrHPRT1 B TALEN target site. Using a similar MhAX cassette structure to that described above for external mutation deposition (FIG. 7A), we designed a new flanking μH ‘TAAGAGATATTGT’ which contained the 312C>A Munich mutation centrally (double underline) and an additional silent mutation 306G>T at the 5′ end of the μH (single underline) that generated an AflII restriction site exclusively for diagnostic purposes (FIG. 10A). The overlap in HPRT1 homology was therefore shifted to accommodate the mutation position (FIG. 10A and FIG. 11). In order to recapitulate the phenomenon observed with imperfect repair of μ5W3 (FIG. 1), we generated two targeting vectors in which the 312C>A patient mutation in the μH was either symmetrical (bilateral), or asymmetrical (unilateral, such that the downstream homology is ‘TAAGAGCTATTGT’) (FIG. 10). Bilaterally encoded mutations were hypothesized to be deposited in 100% of clones, while unilaterally encoded mutations would be deposited in only a fraction of clones. Both μH contained the diagnostic AflII 306G>T mutation. We took no efforts to disrupt the endogenous μ5W3, as both μH components were shifted into the left homology arm, and therefore not expected to affect targeting or excision. Finally, we included a constitutively expressed CAG: :mCherry reporter gene to improve the enrichment of cassette-excised iPSCs. AvrHPRT1_B TALENs were again employed to stimulate gene targeting in 1383D6 iPSCs. Clones were screened by Southern blot (FIG. 11D), PCR amplification followed by AflII cleavage (FIG. 11E) and junction sequencing (data not shown), mCherry expression by FACS (FIG. 10B), as well as sensitivity to HAT and resistance to 6-TG (FIG. 10B) before proceeding with excision.

Excision was induced by transfection of targeted clones 033-U-45 (unilateral) and 033-B-43 (bilateral) with pX-EGFP-g1, producing mCherry negative populations at a rate of 1.9% and 1.4% for 033-U-45 and 033-B-43, respectively (FIG. 12). mCherry negative cells were FACS sorted to >98% purity and replated at clonal density with or without HAT selective pressure. Clonal isolation and metabolic screening revealed that certain iPSC lines displayed a reversal of 6-TG and HAT resistance, indicating normal HPRT, while others displayed sensitivity to both drugs (FIG. 10B). Under HAT selection 033-B-43 yielded no clones, suggesting either a failure to repair or a phenotypic effect of the 312C>A mutation (FIG. 10C). On the other hand, 033-U-45 generated iPSC colonies under HAT selective pressure which all achieved scarless excision but represented deposition of the Silent 306G>T mutation exclusively (49/49), indicating either a repair bias or phenotypic sensitivity for HPRT1^{312A} clones to HAT.

Excision, FACS enrichment, and colony formation in the absence of selective pressure produced scarlessly engineered clones (FIG. 10C). As observed for μ11 (FIG. 7E), clones that repaired via NHEJ generated various indel mutations comprised of eGFP sgRNA1 breakpoints and retention of flanking μH. Amongst clones with bilateral μH, 2.5% (5/204) excised scarlessly, and all clones bore both the 306T Silent and 312A Munich mutations. Clones from unilateral μH excised scarlessly at a rate of 6.6% (14/211). Importantly, 9/14 clones bore both the Silent and Munich mutations, while the remainder (5/14) carried only the Silent mutation (FIG. 10C and D), indicating that we could reproduce the stochasticity of MMEJ outcomes by intentionally engineering imperfect homology. Amongst the correctly excised clones, both FACS analysis for mCherry (FIG. 10B) and Southern blotting (FIG. 10E) with an internal transgene probe again provided evidence that marker genes do not re-insert into the genome at any detectable rate. Thus, our data proves that MMEJ through imperfect μH can be applied to the simultaneous generation of diseased and the associated normal isogenic iPSC clones handled under equivalent experimental conditions.

### Phenotypic Analysis of Engineered HPRT_{Munich} Mutations

Finally, we set out to examine the phenotypic consequences of HPRT engineering and assess clonal variation. HPRT enzymatic activity is required for the conversion of hypoxanthine to inosine monophosphate (IMP) in the purine salvage pathway (FIG. 13A). When de novo synthesis of purines is blocked by HAT medium (hypoxanthine, aminopterin, thymidine) in culture, cells must rely wholly on purine salvage for DNA synthesis. During the MhAX procedure, HAT enrichment selectively eliminated HPRT^{306T/312A} clones in favor of HPRT^{306T} clones (FIG. 10C). However, under normal iPSC maintenance conditions, no difference in morphology or growth rate was noted between normal, mutant, or isogenic control clones. We therefore examined the proliferation of engineered iPSC clones under HAT selection. Within 24 hrs of initiating HAT treatment, knockout HPRT^{delta17A} and 033-U-45 were completely eliminated, while HPRT^{306T/312A} iPSCs showed delayed growth by cell number (d3, FIG. 13B). This decline was associated with a profound change in cell morphology (FIG. 13B, right), and complete cell death by 72 hrs. Interestingly, unlike HPRT^{delta17A} and 033-U-45 knockout iPSCs, HPRT^{306T/312A} iPSCs also retained sensitivity to 6-TG (20 μM, FIG. 10B), yet similar to the HAT response, cell death was delayed when compared to 1383D6 or HPRT^{306T} (data not shown). These data suggest that HPRT306T/312A retain a limited ability to salvage guanine ultimately leading to 6-TG-induced toxicity, yet overall purine salvage in the absence of de novo synthesis is insufficient for DNA replication and cell growth.

Pathologically, reduced HPRT function results in high levels of hypoxanthine, and the conversion of excess hypoxanthine into uric acid (FIG. 13A) which can accumulate in the joints and tendons causing inflammatory arthritis, or more severely in kidney stones or urate nephropathy. In vitro assays using hyperuricemia patient cell lysates indicated that, while intracellular levels of HPRT_{Munich} protein are found to be normal (Wilson et al., J Biol Chem 256, 10306-10312, 1981; Wilson et al., 1982), the mutation results in an enzyme with abnormal hypoxanthine catalytic activity (Wilson and Kelly, 1984). Accordingly, while HPRT protein was undetectable in Western blot analysis of lysates from HPRT^{delta17A}and 033-U-45 knockout iPSC lines, yet three clones each of HPRT^{306T} or HPRT306T/312A revealed protein expression levels comparable to that of 1383D6 (FIG. 13C). In order to evaluate the metabolic status of HPRT_{Munich} in HPRT^{306T/312A} iPSCs, we performed capillary-electrophoresis mass spectrometry (CE-MS) to detect ionic metabolites in spent cell culture media (Wakayama et al., 2015)). Levels of both hypoxanthine and guanine were elevated in knockout iPSCs as compared to 1383D6 (FIG. 13D), as predicted for dysfunction of HPRT-mediated purine salvage. While HPRT^{306T} clones had metabolic profiles resembling 1383D6,HPRT^{306T/312A} iPSCs accumulated both hypoxanthine and guanine, but to a lesser extent than HPRT^{delta17A}or 033-U-45 knockouts. These data are consistent with a low-level salvage of guanine and hypoxanthine, rather than a complete loss of function. As such, we have generated a unique iPSC model of an HPRT1 coding-region variant, using the MhAX technique to scarlessly and stochastically deposit disease-relevant or control point mutations.

### Parameters Affecting MMEJ Cassette Excision

In order to explore the effects of increasing μH length on MMEJ efficiencies, we developed a plasmid-based MMEJ assay analogous to our cassette design used to generate the HPRT_{Munich} allele. We flanked a chloramphenicol/ccdB positive/negative bacterial selection cassette with eGFP-1 (ps1) protospacers and inserted it into a luciferase expression vector with flanking μH of increasing length from 0-50 bp (FIG. 14a, b). Following transfection into HEK293T cells, a positive correlation between μH length and luciferase activity was observed, suggesting an improved rate of MMEJ with increasing μH length (FIG. 14b). Recovery of Kan^{R} cassette-excised plasmids in a ccdB-sensitive bacterial host revealed similar colony numbers across all μH lengths tested (data not shown), reflecting a constant efficiency for psi cleavage across the MMEJ plasmid series. Sequencing of the μ0 junctions from bacterial colonies revealed a consistent pattern of NHEJ, while μ20 junctions revealed perfect MMEJ-mediated repair in 6.25% of Kan^{R} clones (2/32). Thus, in concordance with luciferase activity, increasing μH length improved MMEJ repair over NHEJ.

Precise cassette excision by MMEJ from an extrachromosomal plasmid in HEK293T cells may not accurately reflect cassette excision from the iPS cell genome. We therefore established a chromosomal assay at the HPRT locus where MMEJ results in recovery of HAT resistance, along with the deposition of three synonymous mutations disrupting μ5A3 (c.303A>G, c.304C>T, and c.306G>A). Using TALEN, MhAX cassettes flanked by μH of 11 bp or 29 bp in length were targeted to HPRT1 exon3 (FIG. 14c). Puro^{R} clones were screened by PCR and Southern blot as before, and verified as 6-TG^{R} and HAT^{S}, while flow cytometry revealed constitutive and uniform expression of mCherry in all correctly targeted iPSCs (data not shown). As expected, mCherry negative fractions were similar between the two constructs, indicating that Cas9 cleavage at psi protospacers and cassette excision rates were not affected by μH length. However, mCherry negative cells from μ29 excision gave rise to higher numbers of HAT^{R} colonies (FIG. 14d), suggesting enhanced scarless repair by MMEJ. Genotyping of HPRT alleles from μ11 and μ29 mCherry negative populations (without HAT enrichment) revealed a ˜4-fold increase in scarless repair and mutation deposition (7.8% vs˜35% avg.), similar to the fold-change observed in the plasmid assay (FIG. 14b). Thus, increasing the length of μH improves scarless cassette excision from human iPSC chromosomes.

Evidence from DSBR in yeast (PMID:17483423) and mouse ESCs (PMID:9418857) suggests that the presence of long heterology (non-homologous sequence from the end of DSBs until the start of homology) can negatively impact MMEJ or HDR repair rates. We tested this parameter by simply inverting the ps1 protospacers, such that their PAMs were placed proximal to the selection cassette, leading to a 17 bp heterology on either end compared to 6 or 7 bp generated in the PAM-distal orientation used thus far (FIG. 14e). Cassette excision rates as measured by mCherry-negative cell fractions from PAM-distal or inverted protospacers were similar, indicating that orientation itself does not affect Cas9 cleavage. Although indel-free sequences with engineered synonymous mutations could be enriched in HAT-selected populations from either protospacer orientation, MMEJ repair rates were impeded by elongated heterology as indicated by a reduction in overall HAT^{R} colony formation (FIG. 14f). Conversely, public and empirical data suggests that MMEJ fidelity could be further enhanced by deliberately selecting μH ends which contribute endogenous sequence to the engineered protospacers. Based on these results, subsequent MhAX experiments employed elongated μH and maintained a PAM-distal orientation. Biallelic modification of the APRT locus

Many disease-causing mutations show autosomal recessive inheritance. We thus set out to demonstrate scarless biallelic modification using the MhAX method. For this purpose, we chose to engineer the adenosine phosphorybosyl transferase (APRT) enzyme, which is required for the synthesis of adenosine monophosphate (AMP) from adenine. The APRT*J mutation (c.407T>C; rs104894507; M136T) results in partial enzyme deficiency causing a buildup of 2,8-dihydroxyadenine (2,8-DHA) crystals, often leading to kidney stone formation or more severely, kidney failure (Kamatani et al., 1990). Although the APRT*J mutation is prevalent in Japanese patients with urolithiasis (79%), an in vitro iPSC model of the APRT*J mutation remains to be generated. Employing a gene-trap MhAX cassette flanked by PAM-distal eGPF-1 protospacers (FIG. 15a), we engineered a flanking 32 bp μH:

[Chem. 1]
GTACCACGAACGCTGCCTGTGAGCTGCTGGGC

in which a synonymous c.402A>T mutation (single underline) generating a diagnostic Acc65I restriction site was present bilaterally, while the APRT*J mutation (double underline) was present unilaterally. In order to reduce random integration of the donor vector backbone, we employed negative selection for GFP fluorescence (FIG. 15a, PMID:16258059). CRISPR sgRNAs overlapping the mutation sites in APRT exons were screened using T7E1 digestion and directly in APRT gene targeting. APRT sgRNA-2 was selected for superior performance in both assays. Puromycin-resistant mCh^{pos}/GFP^{neg} iPSC clones were identified by microscopy, picked, and screened for correct targeting by genomic PCR, junction sequencing, Southern blot, and flow cytometry. Mean fluorescence intensity of mCherry showed a bimodal distribution which was linked in a copy number-dependent manner, as verified by genotyping of hetero- and homozygously targeted clones (FIG. 16).

Three each of hetero- and homozygously targeted clones were subjected to selection marker excision via transfection of pX-eGFP-1. Excision rates were consistently higher for heterozygous (6.7% avg.) versus homozygous (3.3% avg) targeted clones (FIG. 15e and data not shown), reflecting the requirement for one or two copies of the selection marker to be removed from the genome. Excised mCh^{neg} populations were isolated by FACS, from which the spectrum of alleles was analyzed by genomic PCR. Expectedly, unmodified normal alleles composed approximately half of the sequences detected in excised populations from heterozygous targeted clones. Scarless excision of the cassette occurred at an average rate of 30% amongst heterozygous clones. Interestingly, homozygous targeted clones showed a relative increase in NHEJ alleles, leading to an overall reduced average rate of 13% scarless excision. Unilateral μH was again observed to stochastically generate both silent and pathogenic allele types.

Populations of mCh^{neg} cells were plated for clonal isolation and genotyping. To ensure the identification of both alleles, we included a neighboring heterozygous SNP (rs8191489, G/C) from intron3 within the PCR amplicon (data not shown), and employed TIDE analysis to decompose heterozygous repair events. The diploid genotypes of all clonally isolated iPSCs are summarized in FIG. 15g. Scarless excision rates in the heterozygously targeted clone EP052-2-2 were similar to that predicted from population analyses (32.2%, FIG. 15g). Homozygous clone EP052-2-11 gave rise to 9/160 (5.6%) excised clones with scarless biallelic modification, representing homozygous and compound heterozygous genotypes (FIG. 15g). Sequence decomposition by TIDE revealed that an additional 18 clones categorized as NHEJ underwent scarless excision of the other allele, such that the overall allelic rate of MhAX fidelity (16.9%) was in agreement with our initial population analysis.

Biallelically engineered APRT*J clones were selected and correct gene editing was further confirmed using Southern blot and an Acc65I RFLP assay (FIG. 15c, d). We phenotyped APRT*J iPSC clones by testing their resistance to 2,6-diaminopurine (DAP), a toxic purine analogue (PMID:3837181). Parental 1383D6 and homozygous Silent/Silent mutants displayed severe drug sensitivity to 10 ug/mL DAP treatment within just 24 hrs. Heterozygous targeted or APRT*J/Silent cells had minor resistance to DAP but were also eliminated within 48 hrs, while homozygous targeted and APRT*J/APRT*J cells were completely resistant to DAP treatment, verifying a functional change in cellular metabolism as a result of APRT knockout or gene editing.

### ‘Liquid’ Modification of the APRT Locus Generates an Isogenic Allelic Series

With the goal of expediting the scarless gene editing process in iPSCs, we chose to exploit the high fidelity of gene-trap targeting with copy-number dependent transgene expression and fluorescent counter-selection of random targeting events by FACS. APRT gene targeting was carried out as described above (FIG. 15), however instead of clonal isolation and screening of targeted intermediates, entire Puro^{R} populations were harvested in bulk and subjected to FACS to isolate mCh^{pos}/GFP^{neg} iPSCs (FIG. 17a, b). We further separated the mCh^{pos} population into mCh^{low} (52.9% of total) and mCh^{high} (15.5% of total) (FIG. 17b) in order to enrich for heterozygous or homozygously targeted cells (FIG. 15/SX), respectively. Cassette excision was more efficient from the mCh^{low} than mCh^{high} (7.0 vs 2.6%) bulk population (FIG. 17b), consistent with excision one or two transgene copies from heterozygous or homozygously targeted clones (FIG. 15).

We first performed genotyping analyses on the two resulting excised populations, classifying alleles into 3 categories: non-targeted, which includes normal and indel alleles (generated during gene targeting); NHEJ, which arise during repair of cassette excision (distinguished from indels as they retain engineered sequences); and MMEJ, which contain the pathogenic and/or silent mutations (FIG. 17c). Notably, the mCh^{low} population contained more frequent indels, while the mCh^{high} population was biased toward NHEJ, validating FACS enrichment of mono- or biallelically-targeted cells, but also revealing the potential of APRT-sgRNA2 to elicit error-prone repair of DSBs. Excluding normal and indel alleles, the fidelity of scarless repair was slightly higher for the mCh^{low} than mCh^{high} (26.5 vs 22.7%) population. A similar process of FACS-based targeting and excision was performed for the HPRT_{Munich} allele (FIG. 18), which gave rise to scarless gene edited clones at a rate similar to that observed previously for cloned intermediates (5.6 vs 6.6%). Finally, we performed clonal isolation and analysis of APRT*J alleles from the bulk excised populations. Thus, our HPRT and APRT gene editing approach demonstrates that engineered MMEJ through imperfect μH can simultaneously generate both diseased and normal isogenic iPSC clones handled under equivalent experimental conditions (FIG. 17e).

Alternate sgRNAs for MhAX Cassette Excision

We screened a series of candidate sgRNAs predicted to have low off-target sites in the human genome (FIG. 19). The candidate list included the sgRNA targeting the GFP gene of A.victoria which we had already demonstrated to be active for MhAX, one sgRNA targeting zebrafish tiall (Hwang et al., 2013) which was recently used to stimulate endogenous gene tagging through NHEJ in human near-haploid HAP1 cells (Lackner et al., 2015), and PITCh, a completely artificial sgRNA sequence used for MMEJ-assisted gene knock-in in human HEK293T cells (Nakade et al., 2014).

### REFERENCES

Bae, S. et al. (2014) Nature methods 11, 705-706.
Brinkman, E. K. et al. (2014) Nucleic acids research 42, e168.
Capecchi, M. R. (2005) Nature reviews Genetics 6, 507-512.
Cariello, N. F. et al. (1988) Am J Hum Genet 42, 726-734.
Cermak, T. et al. (2011) Nucleic acids research 39, e82.
Chen, Y. T., and Bradley, A. (2000) Genesis 28, 31-35.
Christian, M. et al. (2010) Genetics 186, 757-761.
Davis, R. P. et al. (2008) Nature protocols 3, 1550-1558.
Doench, J. G. et al. (2016) Nature biotechnology 34, 184-191.
Doench, J. G. et al. (2014) Nature biotechnology 32, 1262-1267.
Firth, A. L. et al. (2015) Cell reports 12, 1385-1390.
Fu, Y. et al. (2014) Nature biotechnology 32, 279-284.
Hockemeyer, D., and Jaenisch, R. (2016) Cell stem cell 18, 573-586.
Jinek, M. et al. (2012) Science 337, 816-821.
Kim, H., and Kim, J. S. (2014) Nature reviews Genetics 15, 321-334.
Kim, S. I. et al. (2016) Methods Mol Biol 1357, 111-131.
McVey, M., and Lee, S. E. (2008) Trends in genetics: TIG 24, 529-538.
Meier, I. D. et al. (2010) FASEB journal: official publication of the Federation of American Societies for Experimental Biology 24, 1714-1724.
Nakade, S. et al. (2014) Nature communications 5, 5560.
Nakagawa, M. et al. (2014) Scientific reports 4, 3594.
Oceguera-Yanez, F. et al. (2016) Methods 101, 43-55.
Ochiai, H. et al. (2010) Genes Cells 15, 875-885.
Okita, K. et al. (2010) Nature protocols 5, 418-428.
Okita, K. et al. (2011) Nature methods 8, 409-412.
Ramirez-Solis, R. et al. (1992) Anal Biochem 201, 331-335.
Ran, F. A. et al. (2013) Nature protocols 8, 2281-2308.
Sakuma, T. et al. (2013) Genes Cells 18, 315-326.
Sakuma, T. et al. (2013) Scientific reports 3, 3379.
Sakuma, T., and Woltjen, K. (2014) Dev Growth Differ 56, 2-13.
Takahashi, K. et al. (2007) Cell 131, 861-872.
Thomson, J. A. et al. (1998) Science 282, 1145-1147.
Tomoda, K. et al. (2012) Cell stem cell 11, 91-99.
Villarreal, D. D. et al. (2012) PLoS genetics 8, e1003026.
Wakayama, M. et al. (2015) Methods Mol Biol 1277, 113-122.
Wilson, J. M. et al. (1981) J Biol Chem 256, 10306-10312.
Wilson, J. M. et al. (1982) J Clin Invest 69, 706-715.
Wilson, J. M., and Kelley, W. N. (1984) J Biol Chem 259, 27-30.
Ye, L. et al. (2014) Proceedings of the National Academy of Sciences of the United States of America 111, 9591-9596.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

In addition, the contents disclosed in any publication cited herein, including patents and patent applications, are hereby incorporated in their entireties by reference, to the extent that they have been disclosed herein.

This application is based on US provisional patent application No. 62/370,047, the contents of which are incorporated in full herein.

1. A method of producing a cell having a scarless genome sequence wherein an exogenous nucleic acid sequence inserted into a targeted region in the genome is completely excised, wherein the exogenous nucleic acid sequence comprises a nucleic acid sequence homologous to a genome sequence in the targeted region at each end and one or more sequence-specific nuclease-recognizing site(s) between the two homologous nucleic acid sequences, and wherein the method comprises:
(1) introducing the sequence-specific nuclease or a nucleic acid encoding the same into a host cell having a genome sequence into which the exogenous nucleic acid sequence is inserted; and
(2) culturing the cell obtained in step (1),
thereby causing double-strand break at the sequence-specific nuclease-recognizing site(s) and the subsequent microhomology-mediated end joining or single-strand annealing between the resulting broken ends that contain the homologous nucleic acid sequences to generate a cell having a scarlessly reverted genome sequence in which the exogenous nucleic acid sequence is completely excised from the targeted region.

2. The method according to claim 1, wherein the exogenous nucleic acid sequence comprises two or more sequence-specific nuclease-recognizing sites and two of them are located substantially adjacent to the two homologous nucleic acid sequences, respectively, and an exogenous gene is inserted between the two sequence-specific nuclease-recognizing sites.

3. The method according to claim 2, wherein the exogenous gene is a selectable marker gene.

4. The method according to claim 1, wherein either or both of the homologous nucleic acid sequences have a mutation in the corresponding endogenous genome sequence.

5. The method according to claim 4, wherein both of the homologous nucleic acid sequences have the same mutation, thereby generating a cell having a genome sequence with the mutation in the targeted region.

6. The method according to claim 4, wherein either of the homologous nucleic acid sequences has a mutation, thereby simultaneously generating a cell having a genome sequence with the mutation in the targeted region and an isogenic cell without the mutation.

7. The method according to claim 1, wherein the sequence-specific nuclease is a Zinc-finger nuclease (ZFN), a transcription activator-like effector nuclease (TALEN) or a clustered regulatory interspaced short palindromic repeats/CRISPR-associated protein (CRISPR/Cas).

8. The method according to claim 1, wherein the host cell is obtained by
introducing into a cell a nucleic acid comprising the exogenous nucleic acid sequence and, at both ends thereof, genome sequences flanking both ends of a genome sequence homologous to the homologous nucleic acid sequences, respectively,
thereby inserting the exogenous nucleic acid sequence into the targeted region of the host genome by homologous recombination.

9. The method according to claim 8, wherein either or both of the flanking genome sequences have a mutation in the corresponding endogenous genome sequence, thereby generating a cell having a genome sequence with the mutation in the flanking genome sequence(s).

10. The method according to claim 8, wherein the homologous recombination is mediated by sequence-specific double-strand break at a sequence-specific nuclease-recognizing site in each of the flanking genome sequences.

11. The method according to claim 10, wherein the sequence-specific nuclease is ZFN, TALEN or CRISPR/Cas.

12. The method according to claim 1, wherein the host cell is an embryonic stem cell or an induced pluripotent stem cell.

13. The method according to claim 1, wherein the targeted region comprises a site whose mutation causes a disease.

14. An isolated nucleic acid comprising:
(a) two nucleic acid sequences homologous to a targeted region in a host genome, wherein the 3′ end of one of the nucleic acid sequences and the 5′ end of the other nucleic acid sequence overlap; and
(b) one or more sequence-specific nuclease-recognizing site(s) between the two nucleic acid sequences of (a).

15. The nucleic acid according to claim 14, wherein the exogenous nucleic acid sequence comprises two or more sequence-specific nuclease-recognizing sites and two of them are located substantially adjacent to the two nucleic acid sequences of (a), respectively, and an exogenous gene is inserted between the two sequence-specific nuclease-recognizing sites.

16. A kit comprising:
(a) the nucleic acid of claim 14; and
(b) one or more kinds of sequence-specific nuclease(s) specifically recognizing the sequence-specific nuclease-recognizing site(s) contained in the nucleic acid of (a), or nucleic acid(s) that encode the same.

17. The kit according to claim 16, wherein the sequence-specific nuclease is ZFN, TALEN or CRISPR/Cas.
